SEQUENCE LISTING (22 sequences)

SEQ ID NO: 1 - protein, 164 aa, Arabidopsis thaliana
  JUL1 protein
MSRPGDWNCRSCSHLNFQRRDSCQRCGDSRSGPGGVGGLDFGNFGGRAMSAFGFTTGSDVRPGDWYCTVGNCGTHNFASRSTCFKCGTFKDETGAGGGGGGIGGPAMFDADIMRSRVPGNGGRSSWKSGDWICTRIGCNEHNFASRMECFRCNAPRDFSNRTSF

SEQ ID NO: 2 - protein, 170 aa, Arabidopsis thaliana
  JUL2 protein
MNRPGDWNCRLCSHLNFQRRDSCQRCREPRPGGISTDLLSGFGGRPVSSSFGFNTGPDVRPGDWYCNLGDCGTHNFANRSSCFKCGAAKDEFSCSSAAATTGFMDMNVGPRRGLFGFGGSSSGGGGTGRSPWKSGDWICPRSGCNEHNFASRSECFRCNAPKELATEPPY

SEQ ID NO: 3 - DNA, 495 bp, Arabidopsis thaliana
  JUL1 cDNA
atgagcagacccggagattggaactgcaggtcatgcagccatctcaacttccagcgccgtgactcttgccagcgatgcggtgactctcgttccggccccggtggagttggtggcttagactttggtaatttcggtggcagagccatgtctgctttcggattcaccaccggctccgacgttcgtcccggtgattggtactgcaccgtgggaaactgcgggacacacaacttcgccagtcgctccacctgcttcaaatgcggcactttcaaggacgagaccggcgctggaggcggaggtggtggcatcggcggtccggccatgtttgacgccgacattatgcggtctagagtccccggtaacggtggtcgctctagctggaaatccggcgactggatttgcactaggattggttgcaatgagcataactttgcaagcagaatggaatgcttcaggtgcaatgcaccaagggacttcagcaacagaacctctttctaa

SEQ ID NO: 4 - DNA, 513 bp, Arabidopsis thaliana
  JUL2 cDNA
atgaataggccgggagattggaactgcagattgtgtagccacctcaacttccagaggagggattcatgccaacgttgtagagagcctagaccgggcgggatcagtaccgatttactcagcggttttggtggccgtccggttagtagctccttcggtttcaacaccgggcccgatgtgcgacccggggattggtattgcaaccttggggattgtgggacacataattttgccaataggtccagttgtttcaagtgtggtgccgcaaaagatgagttttcatgctcaagtgctgctgcaacaaccgggtttatggacatgaatgttggtccgagacgtggcctttttggttttggcggcagcagtagtggtggtggtggtacgggccgttctccttggaaatctggagattggatttgcccaaggtcaggctgtaacgaacataacttcgcaagcaggtcagagtgtttcaggtgtaacgcaccaaaggaacttgccaccgaaccaccctattag

SEQ ID NO: 5 - DNA, 20 bp, Artificial Sequence
  SEOR1(AT3G01680)_Forward
tctcacggccttggttcatc

SEQ ID NO: 6 - DNA, 20 bp, Artificial Sequence
  SEOR1(AT3G01680)_Reverse
atcgaccccaaccaagttcc

SEQ ID NO: 7 - DNA, 20 bp, Artificial Sequence
  NEN4(AT4G39810)_Forward
cggtaggatttgggcaggtc

SEQ ID NO: 8 - DNA, 21 bp, Artificial Sequence
  NEN4(AT4G39810)_Reverse
ccaagaccaaaataggcagcc

SEQ ID NO: 9 - DNA, 20 bp, Artificial Sequence
  HB8(AT4G32880)_Forward
gctcgctggatctctcactc

SEQ ID NO: 10 - DNA, 20 bp, Artificial Sequence
  HB8(AT4G32880)_Reverse
tcttgaagtgccaccaacgt

SEQ ID NO: 11 - DNA, 20 bp, Artificial Sequence
  TDR(AT5G61480)_Forward
tctcgtcggaaaaccttgca

SEQ ID NO: 12 - DNA, 20 bp, Artificial Sequence
  TDR(AT5G61480)_Reverse
accaccgtcgactctgtttc

SEQ ID NO: 13 - DNA, 20 bp, Artificial Sequence
  IRX3(AT5G17420)_Forward
caaagggtcctaaacgtcca

SEQ ID NO: 14 - DNA, 20 bp, Artificial Sequence
  IRX3(AT5G17420)_Reverse
atggatgattgcccaaatgt

SEQ ID NO: 15 - DNA, 20 bp, Artificial Sequence
  XCP1(AT4G35350)_Forward
tggagaaagaaaggcgctgt

SEQ ID NO: 16 - DNA, 20 bp, Artificial Sequence
  XCP1(AT4G35350)_Reverse
atgagacctccattgcagcc

SEQ ID NO: 17 - DNA, 22 bp, Artificial Sequence
  TUB4(AT5G44340)_Forward
gctaatcctacctttggtgatc

SEQ ID NO: 18 - DNA, 20 bp, Artificial Sequence
  TUB4(AT5G44340)_Reverse
cagctttccacggaacacag

SEQ ID NO: 19 - DNA, 18 bp, Artificial Sequence
  GFP_Forward
agcaagggcgaggagctg

SEQ ID NO: 20 - DNA, 18 bp, Artificial Sequence
  GFP_Reverse
gcacgccgtaggtgaagg

SEQ ID NO: 21 - DNA, 17 bp, Artificial Sequence
  YFP_Forward
gcaagggcgaggagctg

SEQ ID NO: 22 - DNA, 17 bp, Artificial Sequence
  YFP_Reverse
gcactgcaggccgtagc

(19) United States
(12) Patent Application Publication
(10) Pub. No.: US 2021/0115458 A1
(43) Pub. Date: Apr. 22, 2021

(54) COMPOSITION FOR INCREASING NUTRIENT STORAGE CAPACITY OF PLANT NUTRIENT STORAGE TISSUES, CONTAINING JULGI PROTEIN EXPRESSION OR ACTIVITY INHIBITOR

(71) Applicant: POSTECH ACADEMY-INDUSTRY FOUNDATION, Pohang-si (KR)

(72) Inventors: Il Doo HWANG, Pohang-si (KR); Hyun Woo CHO, Cheongju-si (KR); Ho Young NAM, Ulsan (KR); Hyun Seob CHO, Gumi-si (KR)

(21) Appl. No.: 17/041,038

(22) PCT Filed: Mar. 13, 2019
(86) PCT No.: PCT/KR2019/002907

§ 371 Date: Sep. 24, 2020

(30) Foreign Application Priority Data
Mar. 28, 2018 (KR) 10-2018-0035534
Nov. 27, 2018 (KR) 10-2018-0148550

(51) Int. Cl. C12N 15/82

(52) U.S. Cl. CPC C12N 15/8223; C12N 15/8213; C12N 15/8218

(57) ABSTRACT

The present invention relates to a composition for increasing the sink strength of sink tissues, containing an inhibitor of the expression or activity of a JULGI protein or a protein having 80% or more homology with the JULGI protein; a method for increasing the sink strength of sink tissues of plants by using the composition; and a plant body of which the sink strength is increased through the method. A plant body is treated with a composition containing an inhibitor of the expression or activity of a JULGI protein or a protein having 80% or more homology with the JULGI protein, according to the present invention, such that the development of the phloem, which is an organ serving the most important role in the movement of photosynthates and eventual storage thereof in sink tissues in starch and sugar forms in plants, can be enhanced so as to increase the sink strength of sink tissues in plants, and thus the productivity of various crops including vascular plants in which JULGI is conservatively expressed can be promoted. In addition, unlike conventional methods, a method for increasing the sink strength of plants, according to the present invention, can overcome conventional limitations by being a method that produces higher value-added crops into which no exogenous gene is introduced and which are free of GMO problems.

### TECHNICAL FIELD

The present invention relates to a composition for increasing the sink strength of a sink tissue of a plant, which includes an inhibitor of the expression or activity of a JULGI protein, a method of increasing the sink strength of plants using the composition, and a plant body in which the sink strength increases by the method.

### BACKGROUND ART

The amount of damage from an abnormal global climate is increasing due to the increase in atmospheric carbon dioxide concentration, and the Paris Agreement for the regulation of CO₂ emission, adopted by consensus in 2015 by 195 countries, indicates how important the regulation of an atmospheric CO₂ concentration is for human survival. The process of converting CO₂ to a carbon compound through photosynthesis is a key mechanism that constitutes the global carbon cycle, and a primary production process that converts light energy obtained from the sun to organic energy in the ecosystem of the earth. Moreover, understanding and application of the fundamental principles of the development and growth of plants, which are independent nutritional organisms, are an essential part of the survival of the human race, not the survival of an individual human being. Therefore, conventionally, as a strategy for promoting carbon assimilation efficiency and crop yield, methods of increasing the activity of an enzyme involved in photosynthesis or promoting the expression of a carrier protein of a photosynthate have been tried. In addition to these methods, a method of promoting the transport capacity of phloem, which is a factor that inhibits the transport of a photosynthate may also be a significant strategy.

Phloem, a living conduit in vascular plants, plays a critical role in the development of plants as a transport pathway of macromolecules such as photosynthates, hormones, mRNA, and proteins, and particularly mainly serves to develop and regulate sink organs that store and use photosynthates. Phloem differentiation involves irreversible cellular reprogramming from meristematic cells called the (pro)cambium, and during this transition, selective elimination of subcellular organelles including the nucleus, cell wall remodelling and cytosolic dilution occur through the regulation of signals for a transcriptional cascade. After the fate of the phloem cells is determined, phloem initial cells are enucleated to develop sieve elements, which are joined together to forma sieve tube. As the cells are losing their transcriptional capacity, post-transcriptional regulation may be necessary to construct the phloem network in plants. However, the post-transcriptional regulatory mechanism that becomes the basis of the phloem differentiation is unknown, and the influence on shaping of a source-sink relationship remains to be elucidated.

Therefore, research on phloem differentiation is actively progressing globally, and knowledge on the mechanism of regulating phloem differentiation is also gradually expanding. More particularly, in 2003, through the discovery of a gene involved in regulating the development of phloem, that is, an ALTERED PHLOEM DEVELOPMENT (APL) gene, research on a phloem differentiation mechanism has been initiated (Bonke et al., 2003 Nature 426:181-186), and recently, a downstream transcription factor NAC45/86 as a downstream regulatory gene of APL and NEN1-4, involved in enucleation occurring in the phloem differentiation, were identified. In addition, major regulators for phloem development, such as OCTOPUS, BIN2, CVP2, CVL1, BRX, BAM3 and CLE45, were identified. However, until now, no gene suitable for artificially regulating phloem development by humans has not been found. For this reason, the present invention is intended to suggest a method of increasing the sink strength of a sink tissue in plants using a gene that can regulate phloem development to ultimately increase the productivity of crops.

### DISCLOSURE

### Technical Problem

In the present invention, a negative regulatory mechanism of suppressing the expression of SUPPRESSOR OF MAX2 1-LIKE4/5 (SMXL4/5) by inducing G-quadruplex formation by binding of a RNA-binding protein, JULGI, having three RanBP2-type zinc finger domains to the 5′ UTR of SMXL4/5, and thereby suppressing phloem differentiation was identified, and therefore, it was confirmed that phloem development may be improved by suppressing the expression or activity of JULGI, and the sink strength of a sink tissue in plants, for example, seeds or fruits, may be enhanced. Thus, the present invention was completed.

Therefore, the present invention is directed to providing a composition for increasing the sink strength of a sink tissue in a plant, which includes an inhibitor of the expression or activity of a JULGI protein or a protein having 80% or more homology with the protein.

The present invention is also directed to providing a method of increasing the sink strength of a sink tissue in a plant, which includes treating a plant body with the composition.

The present invention is also directed to providing a plant body in which the sink strength of a sink tissue in a plant by the above-described method.

However, technical problems to be solved in the present invention are not limited to the above-described problems, and other problems which are not described herein will be fully understood by those of ordinary skill in the art from the following descriptions.

### Technical Solution

In order to achieve the objects of the present invention as described above, the present invention provides a composition for increasing the sink strength of a sink tissue in a plant, which includes an inhibitor of the expression or activity of a JULGI protein or a protein having 80% or more homology with the protein.

In one embodiment of the present invention, the sink tissue may be a seed, fruit, flower, root or tuber.

In another embodiment of the present invention, the JULGI protein may consist of an amino acid sequence represented by SEQ ID NO: 1 or 2.

In still another embodiment of the present invention, the JULGI protein may suppress the expression of SMXL4/5 by binding to the 5′-untranslated region (UTR) of SMXL4/5 mRNA.

In yet another embodiment of the present invention, the suppression of the SMXL4/5 expression may be performed by inducing the formation of RNA G-quadruplex by binding a JULGI protein to the 5′ UTR of SMXL4/5 mRNA.

In yet another embodiment of the present invention, the inhibitor may be a recombinant virus-induced gene silencing (VIGS) vector, which includes a gene encoding a JULGI protein or a protein having 80% or more homology with the protein.

In yet another embodiment of the present invention, the inhibitor may be an RNAi vector, which includes a gene encoding a JULGI protein or a protein having 80% or more homology with the protein.

In yet another embodiment of the present invention, the inhibitor may be a CRISPR/Cas9 vector that edits a gene encoding a JULGI protein or a protein having 80% or more homology with the protein.

In yet another embodiment of the present invention, the gene encoding the JULGI protein may consist of a nucleic acid sequence represented by SEQ ID NO: 3 or 4.

In yet another embodiment of the present invention, the plant may be selected from the group consisting of food crops including rice, wheat, barley, corn, peas, potatoes, red beans, oats, sorghum; vegetable crops including Arabidopsis, Chinese cabbage, radish, red pepper, strawberry, tomato, watermelon, cucumber, cabbage, melon, pumpkin, green onion, onion and carrot; special crops including Ginseng, tobacco, cotton, sesame, sugar cane, beet, Perilla, peanut and rapeseed; fruit trees including apple, pear, jujube, peach, kiwi, grape, tangerine, persimmon, plum, apricot and banana; flowers including roses, Gladiolus, Gerbera, carnations, Chrysanthemum, lilies and tulips; and forage and fodder crops including ryegrass, red clover, orchard grass, alfalfa, tall fescue and perennial ryegrass.

The present invention also provides a method of increasing the sink strength of a sink tissue in a plant, which includes treating a plant body with the composition.

The present invention also provides a plant body in which the sink strength of a sink tissue in a plant increases according to the method.

### Advantageous Effects

In the present invention, a function of negatively regulating the phloem development of a plant body by silencing SMXL5 by inducing G-quadruplex formation by binding of a JULGI protein to the 5′ UTR of SMXL4/5 was identified, and thus it was confirmed that, when the expression or activity of JULGI is inhibited, phloem development is promoted, and the sink strength of sink tissues such as seeds and fruits increases. Accordingly, a plant body is treated with a composition including an inhibitor of the expression or activity of a JULGI protein according to the present invention, so that the development of the phloem, which is an organ playing the most important role in transport of photosynthates in plants and eventual storage in starch and sugar forms in sink tissues, can be promoted and thus the sink strength of the plant sink tissues increases, resulting in promoting the productivity of various crops including vascular plants conservatively expressing JULGI.

In addition, a method of increasing the sink strength of a plant according to the present invention can overcome conventional limits since this method is a method of producing higher-value added crops, which are free of GMO problems, without the introduction of a foreign gene, unlike conventional methods.

### DESCRIPTION OF DRAWINGS

FIG. 1A shows the result of comparative transcriptome analysis of the phloem/cambium regions of Populus tremula and Arabidopsis thaliana, and sucrose regulated genes of Arabidopsis thaliana.

FIG. 1B shows the results of measuring the degree of phloem differentiation and the change in expression levels of the phloem, cambium and xylem marker genes (APL, WOX4 and XCP2, respectively), after silencing of a tobacco gene, Niben101Scf0620g02009 (At3g15680 ortholog), which has three RanBP2-type ZnF domains (TRV-NbJUL).

FIG. 1C shows the result of examining the orthologs of tobacco JUL in 23 different species of plants through phylogenetic analysis.

FIG. 1D shows the results of measuring the degree of phloem differentiation and the expression levels of APL, WOX4, XCP2 marker genes according to JUL1 and JUL2 silencing (JUL1/2 RNAi) or JUL1 protein overexpression (JUL1 OX) in Arabidopsis thaliana.

FIG. 1E shows the results of measuring the expression levels of the phloem, cambium and xylem marker genes (SEOR1, TDR and IRX3) according to the induction period for JUL1/2 RNAi or JUL1 OX to evaluate the role of JUL in the establishment of a vascular system using a vascular cell induction culture system.

FIG. 2A shows three RanBP2-type ZnF domains and a conserved arginine residue required for RNA binding in a JUL1 protein, and the results of indicating the degree of phloem differentiation according to the overexpression of each JUL1 mutant in which the arginine residue is substituted with alanine.

FIG. 2B shows the result of analyzing whether the JUL gene has a general RNA binding capability or sequence specificity using a random pentaprobe (PP) ssRNA library.

FIG. 2C shows the result of analyzing a set of 67 phloem-specific genes assumed to have an RNA G-quadruplex-forming motif to find an in vivo mRNA target of JUL1.

FIG. 2D shows the result of a phylogenetic analysis of the SMXL family known as a significant positive regulator for phloem differentiation in Arabidopsis thaliana.

FIG. 2E shows the result of observing the degree of phloem differentiation after silencing of SMXL5 exclusively conserved in vascular plants in tobacco (TRV-NbSMXL5).

FIGS. 2F to 2H show results of observing phloem tissue and measuring expression levels of phloem, cambium and xylem marker genes (APL and SEOR1/TDR and HB8/IRX3 and XCP1, respectively) in smx14/5 Arabidopsis mutants.

FIG. 3A shows results of performing EMSA using GST-JUL1 in the presence or absence of potassium, to confirm that G-quadruplex formation is induced by direct binding of JUL to the RNA G-quadruplex-forming motif of the SMXL5 5′ UTR.

FIG. 3B shows the result of measuring the dissociation constants of JUL1 and the SMXL5 5′ UTR in the presence or absence of potassium.

FIG. 3C shows the result of analyzing the interaction between the SMXL5 5′ UTR G-quadruplex and a JUL1 protein using a surface binding system.

FIG. 3D shows the result of a reverse transcriptase stop assay for the RNA G-quadruplex structure of the SMXL5 5′ UTR.

FIG. 3E shows the result of the circular dichroism (CD) absorptivity of G-quadruplex-forming motifs in SMXL5.

FIG. 3F shows the result of indicating the binding of JUL1 to the SMXL5 5′UTR in vivo using RNA-immunoprecipitation in protoplasts.

FIG. 4A shows the results of observing JUL and SMXL5 expression patterns in vascular tissues of flowering stems using a system expressing β-glucuronidase (GUS) and YFP under the control of JUL1 and SMXL5 promoters, respectively.

FIG. 4B shows the results of visualizing the cellular distribution of JUL1 and the SMXL5 5′ UTR with fluorescence using a MS2 hairpin/MS2 coat protein-based RNA monitoring system.

FIG. 4C shows the results of measuring the degree of phloem differentiation and expression levels of related marker genes in transgenic lines overexpressing JUL1 fused with a nuclear localization sequence (NLS) or a nuclear export sequence (NES).

FIG. 5A shows the result of observing whether SMXL5 binds to ribosomes in the presence or absence of JUL1.

FIG. 5B shows the result of analyzing whether SMXL5 translation is inhibited by JUL1 by observing the change in GFP expression levels according to the concentration of JUL1 using a GFP reporter expressed under the control of the SMXL5 5′ UTR.

FIG. 5C shows the results of reporter assays for SMXL5 5′ UTR or mutant SMXL5 5′ UTR-fused luciferase (LUC) to indicate the inhibition of SMXL5 translocation according to the concentration of JUL1 (wild type) or mutant JUL1 (RA).

FIG. 5D shows the results of GFP reporter assays to confirm the degree of inhibition of SMXL5 translation according to G-quadruplex formation of the SMXL5 5′ UTR in the presence of JUL1.

FIG. 5E shows the results of GFP reporter and luciferase (LUC) reporter assays to confirm a degree of inhibition of SMXL5 translation according to G-quadruplex formation of the SMXL5 5′ UTR in the presence of JUL1.

FIG. 6A shows the result of observing the expression patterns of SMXL5 in JUL1/2-silenced roots.

FIG. 6B shows the result of analyzing whether SMXL5 is associated with polysomes when JUL1/2 expression is inhibited.

FIGS. 6C and 6D show the results of observing the degree of phloem differentiation according to an SMXL4/5 mutant when JUL is silenced in Arabidopsis thaliana and tobacco.

FIG. 7A shows the experiment performed to trace phloem flow of a phloem symplasmic tracer (CFDA) by treating a plant body with CFDA.

FIG. 7B shows the result of confirming the number of phloem cells and CFDA accumulation in the hypocotyl when JUL1/2 expression is inhibited.

FIG. 7C shows the result of measuring the size and weight of seeds by NbJUL silencing in tobacco (TRV-NbJUL #1, TRV-NbJUL #2).

FIG. 7D shows the result of measuring the degree of gene silencing and the size and weight of seeds when JUL1/2 is silenced in Arabidopsis thaliana (JUL1/2 RNAi).

FIG. 7E shows the result of measuring the total yield and sugar content of tomato flesh according to JUL silencing in tomatoes.

FIG. 8A shows that a single nucleic acid insertion is induced into a 5′ adjacent nucleic acid sequence of a JUL1 gene to knockout the gene.

FIG. 8B shows the result indicating that the phloem highly increases in a JUL1 gene-knockout group (julCRISPR/Cas9jul2), compared with a control (jul2).

FIG. 8C shows the results indicating that the size and weight of seeds increase in a JUL1 gene-knockout group.

FIG. 9 is a diagram showing the result that increased SMXL4/5 translation and resultant increases in phloem development and sink strength are directly related with the regulation of JULGI expression.

### MODES OF THE INVENTION

In the present invention, a negative regulation mechanism of phloem differentiation caused by JULGI was identified, and the regulation of phloem development and the regulation of crop productivity by the regulation of JULGI expression were confirmed.

Therefore, the present invention provides a composition for increasing the sink strength of a sink tissue in a plant, which includes an inhibitor of the expression or activity of a JULGI protein or a protein having 80% or more homology with a homologous protein suggested by a homology assay.

The JULGI protein according to the present invention or a protein having 80% or more homology with the protein includes a JUL1 protein consisting of an amino acid sequence represented by SEQ ID NO: 1, and a JUL2 protein consisting of an amino acid sequence represented by SEQ ID NO: 2, and functional equivalents of the proteins. The “functional equivalent” refers to a protein which exhibits substantially the same physiological activity with the protein represented by SEQ ID NO: 1 or 2, having at least 70% or more, preferably, 80% or more, more preferably, 90% or more, and even more preferably 95% or more sequence homology with the amino acid sequence represented by SEQ ID NO: 1 or 2, as a result of the addition, substitution or deletion of an amino acid. The “substantially the same physiological activity” means the activity for phloem formation in a plant body.

A homologous protein of JULGI suggested by the homology assay is shown in Table 1 below.

TABLE 1

| Species | Name in tree | No. | Gene ID | ID source | Locus | Locus source |
|---|---|---|---|---|---|---|
| Arabidopsis | Arabidopsis | 1 | Q9LW11 | Uniprot | 3: 5314817-5316617 | Ensembl |
| thaliana | | 2 | Q8GWD1 | Uniprot | 5: 8876321-8877904 | Ensembl |
| Brassica | B. rapa | 1 | M4CBM4 | Uniprot | A03: 17339473-17340080 | Ensembl |
| rapa | | 2 | M4DXB1 | Uniprot | A01: 23814143-23814796 | Ensembl |
| | | 3 | M4D046 | Uniprot | A06: 17639554-17640276 | Ensembl |
| | | 4 | M4DVF8 | Uniprot | A02: 24928721-24929455 | Ensembl |
| Eucalyptus | Eucalyptus | 1 | A0A059BST6 | Uniprot | Chr06: 37559529-37560648 | Phytozome |
| grandis | | 2 | A0A059CPS7 | Uniprot | Chr03: 28837096-28838244 | Phytozome |
| | | 3 | A0A059CSU8 | Uniprot | Chr03: 57085574-57086309 | Phytozome |
| Medicago | Medicago | 1 | A0A072UG31 | Uniprot | 4: 2643799-2646573 | Ensembl |
| truncatula | | 2 | G7JB51 | Uniprot | 3: 49624495-49625913 | Ensembl |
| Glycine | Soybean | 1 | C6T4M1 | Uniprot | 8: 17009508-17011639 | Ensembl |
| max | | 2 | C6SXC6 | Uniprot | 7: 2538140-2540048 | Ensembl |
| | | 3 | I1K8W8 | Uniprot | 6: 5362065-5364532 | Ensembl |
| | | 4 | I1JUE6 | Uniprot | 4: 5714874-5717332 | Ensembl |
| Solanum | Potato | | M1ABJ1 | Uniprot | PGSC_DMγ3_219_ch | Solgenomics |
| tuberosum | | | | | 08: 28837851-28839101 | |
| Populus | Popular | 1 | B9GWD5 | Uniprot | 3: 7353724-7355235 | Ensembl |
| trichocarpa | | 2 | A9PB85 | Uniprot | 1: 14100201-14101518 | Ensembl |
| | | 3 | A9P992 | Uniprot | 6: 24868717-24870073 | Ensembl |
| | | 4 | B9IKV1 | Uniprot | 18: 1420333-1421862 | Ensembl |
| Hordeum | Barley | 1 | F2EG93 | Uniprot | Chr6H: 189312796- | Ensembl |
| vulgare | | | | | 189321121 | |
| | | 2 | F2EDY2 | Uniprot | Chr6H: 189312795- | Ensembl |
| | | | | | 189321121 | |
| Oryza | Rice | 1 | Q9SNS0 | Uniprot | 5: 2162738-2165150 | Ensembl |
| sativa | | 2 | Q6Z6E6 | Uniprot | 2: 1420333-1421862 | Ensembl |
| Zea | Maize | 1 | A0A1D6NVS8 | Uniprot | 9: 20521786-20527702 | Ensembl |
| mays | | 2 | B4FHT6 | Uniprot | 5: 114786885-114788112 | Ensembl |
| | | 3 | C0P4Z1 | Uniprot | 4: 236999463-237005338 | Ensembl |
| Sorghum | Sorghum | 1 | C5Z3T1 | Uniprot | Chr10: 2492827-2498546 | Phytozome |
| bicolor | | 2 | Sb04g007110 | Phytozome | Chr04: 7132525-7136914 | Phytozome |
| Ostreococcus | O. | | A4S2N4 | Uniprot | 9: 339159-339585 | Ensembl |
| lucimarinus | lucimarinus | | | | | |
| Selaginella | S. | 1 | D8ST26 | Uniprot | GL377639228833-229684 | Ensembl |
| moellendorffi | moellendorffi | | | | | |
| | | 2 | D8SY24 | Uniprot | GL377652: 363891-367476 | Ensembl |
| cucumis | Cucumber | 1 | A0A0A0K718 | Uniprot | scaffold00929: 24359- | Phytozome |
| sativus | | | | | 25976 | |
| | | 2 | A0A0A0KTI0 | Uniprot | scaffold00919: 2132690- | Phytozome |
| | | | | | 2134447 | |
| | | 3 | Cucsa. 162240 | Phytozome | scaffold01144: 1943572- | Phytozome |
| | | | | | 1945443 | |
| Marchantia | M. | | Mapoly0019s0131 | Phytozome | scaffold_19: 1145977- | Phytozome |
| polymorpha | polymorpha | | | | 1151602 | |
| Sphagnum | S. | | Sphfalx0050s0025 | Phytozome | super_50: 235612-238551 | Phytozome |
| fallax | fallax | | | | | |
| Nicotiana | Tobacco | | Niben101Scf01620g02009 | Solgenomics | Niben101Scf01620: 387421- | Solgenomics |
| benthamiana | | | | | 389889 | |
| solanum | Tomato | | Solyc08g057180 | Phytozome | SL30ch08: 56220160- | Solgenomics |
| lycopersicum | | | | | 56220882 | |

The inventors identified a negative regulation mechanism of JULGI for phloem differentiation with reference to exemplary embodiments, and confirmed that phloem development and the sink strength of a sink tissue increase according to the regulation of the expression of JULGI.

In one embodiment of the present invention, an NbJULGI (NbJUL) gene having three RanBP2-type ZnF domains was confirmed in tobacco, and as a result of investigating homologs of the gene in various plant species, it was observed that the gene is conservatively present in various vascular plants. In addition, as a result of analyzing the effect on phloem formation through the regulation of JUL gene expression, it was confirmed that JUL serves as a negative regulator of phloem differentiation (see Example 2).

In another embodiment of the present invention, as a result of investigating a target of the JUL protein, it was confirmed that the RNA binding activity of a ZnF domain having a RNA-binding arginine residue in the JUL protein, that is, the protein, is essential for the inhibition of phloem development, and it was found that JUL has sequence specificity for target RNA through an experiment using a random pentaprobe ssRNA library. Further, it was confirmed that the G-quadruplex-forming motif of the SMXL4/5 5′ UTR exclusively conserved only in vascular plants is a JUL-specific substrate through a SELEX analysis and a phylogenetic analysis for the SMXL family found thereby (see Example 3).

In still another embodiment of the present invention, as a result of analyzing whether or not to form a G-quadruplex by direct binding between JUL and the RNA G-quadruplex motif of the SMXL5 5′ UTR through various methods, it was confirmed that the G-quadruplex of the SMXL5 5′ UTR directly binds to RanBP2-type ZnF of JUL, RNA G-quadruplex formation is induced at the 5′ UTR of SMXL5 by the above binding, and JUL also binds to the G-quadruplex motif of the SMXL5 5′ UTR in vivo (see Example 4).

In yet another embodiment of the present invention, to specifically identify the action of JUL on SMXL5 mRNA, as a result of monitoring ribosome binding of SMXL5 in protoplasts, it was confirmed that JUL inhibits the binding of an SMXL5 transcript to a ribosome activated for translation through recognition of the 5′ UTR G-quadruplex, and substantially, the expression of a GFP protein under control of the SMXL5 5′ UTR is reduced in a JUL1 dose-dependent manner, and from a result that a decrease in GFP protein expression does not occur in an SMXL5 5′ UTR mutant in which G-quadruplex formation does not occur even in the presence of JUL1, it was confirmed that the direct recognition of the RNA G-quadruplex-forming motif and the stabilization of the G-quadruplex at the SMXL5 5′ UTR induce effective inhibition of JUL1-mediated translation (see Example 5).

In yet another embodiment of the present invention, as a result of investigating the effect of the formation of the 5′ UTR G-quadruplex of JUL-mediated SMXL5 in phloem differentiation, it was confirmed that SMXL5 translation increases by JUL1/2 silencing, through the increase in binding of a polysome to SMXL5 in a JUL1/2-silenced plant body, and as a result of investigating the relationship between JUL and SMXL4/5, it was found that JUL functions upstream of SMXL4/5 in phloem development (see Example 6).

In yet another embodiment of the present invention, as a result of analyzing whether an increase in number of phloem cells relates to phloem transport capacity, it was confirmed that the number of phloem cells increases and the phloem transport capacity is enhanced in a JUL1/2-silenced plant body, and as a result of observing seeds, which are sink tissue in tobacco and Arabidopsis thaliana, it was observed that the size and weight of seeds in the JUL-silenced plant body significantly increase, and it was also confirmed that the total yield and sugar content of tomato fruit significantly increase. Further, also in the case of a JUL1 gene that is knocked out by gene editing, compared with a control, it was confirmed that the phloem is significantly larger, and the size and weight of seeds increase. Therefore, it can be shown that there is a positive relationship between the improvement in phloem differentiation by JUL silencing and the increase in sink strength of a sink tissue (see Examples 7 and 8).

In the present invention, the sink tissue of plants may be a seed, fruit, flower, root or tuber, but the present invention is not limited thereto.

In the present invention, the inhibitor may be a recombinant virus-induced gene silencing (VIGS) vector, a vector for RNAi, or a CRISPR/Cas9 vector, which includes a gene encoding a JULGI protein or a protein having 80% or more homology with the protein. Moreover, a gene editing technique such as ZFN or TALEN may be used, but the type of technique is not limited as long as it can inhibit the expression or activity of the JULGI protein.

In the present invention, a gene encoding the JULGI protein or a protein having 80% or more homology with the JULGI protein may include both of genomic DNA and cDNA, which encode the protein. Preferably, the gene of the present invention may be a JUL1 gene consisting of a nucleic acid sequence represented by SEQ ID NO: 3 or a JUL2 gene consisting of a nucleic acid sequence represented by SEQ ID NO: 4. The nucleic acid sequence represented by SEQ ID NO: 3 is a sequence encoding a protein consisting of the amino acid sequence of SEQ ID NO: 1, and the nucleic acid sequence represented by SEQ ID NO: 4 is a sequence encoding a protein consisting of the amino acid sequence of SEQ ID NO: 2. In addition, a mutant of the nucleic acid sequence is included within the scope of the present invention. Specifically, the gene may include a nucleic acid sequence having 70% or more, more preferably, 80% or more, even more preferably, 90% or more, and most preferably, 95% or more homology with the nucleic acid sequence of SEQ ID NO: 3 or 4. The “% homology” with respect to a polynucleotide is confirmed by comparing two optimally arranged sequences with a comparative region, and a part of the polynucleotide sequence in the comparative region may include an addition or deletion (that is, a gap), compared with a reference sequence (not including addition or deletion) for the optimal alignment of the two sequences.

The VIGS, virus-induced gene silencing, is a type of post-transcriptional gene silencing well known in various plants, fungi, insects, nematodes, fish and rats, and may also be a phenomenon of inhibiting both of the expression and replication of a native gene and a viral genome when a native gene having homology with a viral genome is expressed in an infected plant body in the process of replicating viruses which have invaded a plant body. That is, when a part or all of the specific gene derived from a host is inserted into cDNA of the viral genome, thereby manufacturing infectious RNA, and then the plant body is infected with the infectious RNA, the expression of a target gene is inhibited or lost in the infected host plant body using RNA of the corresponding host as a target. As a result, functions of the target gene may be indirectly estimated. The VIGS mechanism is known as a type of plant defense mechanism mediated by RNA against viruses, and has characteristics of 1) post transcriptional gene silencing 2) RNA conversion and 3) nucleotide sequence specificity.

The term “recombination” used herein refers to cells that replicate a heterologous nucleic acid, express the nucleic acid, or express a peptide, a heterologous peptide, or a protein encoded by a heterologous nucleic acid. The recombinant cells may express a gene or gene fragment, which is not found in a native form of the cells into one of a sense or antisense form. In addition, the recombinant cells may express a gene found in the native-form of cells, but the gene is modified and thus reintroduced into the cells by an artificial means.

In addition, the term “vector” used herein is used to indicate a DNA fragment(s) or a nucleic acid molecule, which is delivered into cells. The vector may replicate DNA, and may be independently reproduced in host cells. A preferable example of the recombinant vector in the present invention is a Ti-plasmid vector which is capable of transferring a part of the vector itself, a so-called T-region, when present in a suitable host such as Agrobacterium tumefaciens, to plant cells. A different type of Ti-plasmid vector is used to transfer a hybrid DNA sequence to current plant cells or protoplasts from which a novel plant in which hybrid DNA is suitably inserted into the genome of a plant can be produced. A particularly preferable type of Ti-plasmid vector is a so-called binary vector. Other suitable vectors which are able to be used to introduce DNA according to the present invention into a plant host may be selected from a viral vector derived from a double-stranded plant virus (e.g., CaMV), a single-stranded virus, and a gemini virus, for example, an incomplete plant virus vector. The vector may be advantageously used when it is difficult to properly transform a plant host. In addition, in the recombinant vector of the present invention, a promoter may be a CaMV 35S, actin, ubiquitin, pEMU, MAS or histone promoter, but the present invention is not limited thereto. The term “promoter” refers to a DNA region upstream from a structural gene, and a DNA molecule to which RNA polymerase binds in order to initiate transcription.

In the present invention, the plant may be selected from the group consisting of food crops including rice, wheat, barley, corn, peas, potatoes, red beans, oats, sorghum; vegetable crops including Arabidopsis, Chinese cabbage, radish, red pepper, strawberry, tomato, watermelon, cucumber, cabbage, melon, pumpkin, green onion, onion and carrot; special crops including Ginseng, tobacco, cotton, sesame, sugar cane, beet, Perilla, peanut and rapeseed; fruit trees including apple, pear, jujube, peach, kiwi, grape, tangerine, persimmon, plum, apricot and banana; flowers including roses, Gladiolus, Gerbera, carnations, Chrysanthemum, lilies and tulips; and forage and fodder crops including ryegrass, red clover, orchard grass, alfalfa, tall fescue and perennial ryegrass, but the present invention is not limited thereto.

In another aspect of the present invention, the present invention provides a method of increasing the sink strength of a sink tissue in a plant, which includes treating a plant body with the composition described above.

In still another aspect of the present invention, the present invention provides a plant body in which the sink strength of a sink tissue in a plant increases according to the method.

The “treating a plant body with the composition” used herein refers to, more preferably, the introduction of DNA into a plant, even more preferably, a transformation method. The transformation method in the present invention may be suitably selected and used by those of ordinary skill in the art according to a known method, and may be selected from, for example, a calcium/polyethylene glycol method for a known protoplast, electroporation of protoplasts, microinjection into a plant element, a (DNA or RNA-coated) particle impact method for various types of plant elements, agroinfiltration, or infection by a (incomplete) virus in Agrobacterium tumefaciens-mediated gene transfer caused by transformation of mature pollen or vesicles. However, in the present invention, more preferably, an Agrobacterium-mediated DNA introduction method may be used.

In yet another aspect of the present invention, the present invention provides a composition for inhibiting translation of SMXL4/5 through RNA G-quadruplex formation, which includes a JULGI protein or a protein having 80% or more homology with the protein.

Hereinafter, to help in understanding the present invention, exemplary examples will be suggested. However, the following examples are merely provided to more easily understand the present invention, and not to limit the present invention.

### EXAMPLES

### Example 1. Experimental Preparation and Methods

1-1. Preparation of Plant Models

In this example, col-0 and WS-2 ecotypes of Arabidopsis thaliana were used as wild type and transgenic lines, respectively. All seeds were germinated in media containing 1/2 Gamborg B5 salt (Duchefa, Netherlands), 1% sucrose and 0.8% phytoagar (pH 5.7) under long-day conditions (16 h light/8 h dark) at 24° C. After a week, the seedlings were transplanted into pots and grown under long-day conditions as described above. Smx15, smx14/5, pSMXL5-YFP and pSMXL5-SMXL5-YFP in smx14/5 lines were provided by Thomas Greb (Heidelberg University, Germany), and jul1(FLAG_293A10; 5′ UTR insertion) and jul2 (GK-268A03-015068; 5′ UTR insertion) were obtained from ABRC. Meanwhile, protoplasts were isolated from fully-expanded leaves of 3- to 4-week-old plants after plants were cultured under short-day conditions (10 h light/14 h dark). Tobacco (N. benthamiana) seeds were sown and cultured under long-day conditions at 26° C. for 5 to 6 weeks, and tomatoes (Solanum lycopersicum cv. Heinz 1706) was cultured under long-day conditions at 22° C. for 5 to 7 weeks.

1-2. Plasmid Construction and Generation of Transgenic Plants

For VIGS analysis, cDNA fragments of NbJUL, S1JUL and NbSMXL5 were cloned into a TRV2 vector. For an RNAi construct, partial sequences of AtJUL1 (AT3G15680) and AtJUL2 (AT5G25490) were amplified and ligated together, and then the ligated product was amplified and cloned into a pCR8/GW/TOPO vector (Invitrogen, USA). The cloned cDNA was amplified using M13 forward and reverse primers, and used for gateway cloning into pK7GWIWG2 (I). For a CRISPR/Cas9 construct, a pHCas9GM-AtU6p vector having the nucleic acid sequence, that is, GGTGATTGGTACTGCACCG, of a region adjacent to 5′ end of AtJUL1 as sgRNA was cloned.

In addition, to tissue-specifically express AtJUL1 and AtJUL2, the 1.5-kb sequence upstream of the translation start site from Arabidopsis thaliana genomic DNA was amplified and cloned into pCXGUS-P.

Meanwhile, for a protoplast reporter assay, the full-length 5′ UTR of SMXL5 and JUL1 were cloned into a plant body expression vector containing GFP, luciferase and HA. The recombinant proteins were produced and purified by cloning the full-length JUL1 and JUL2 cDNA into pGEX5-1 (Promega, USA). The point mutations of JUL1 (JUL1^{R20A}, JUL1^{R80A}, JUL1^{R146A}, JUL1^{R20/80A}, JUL1^{R20/146A}, JUL1^{R80/146A} and JUL1^{R20/80/146A}) and SMXL5 5′ UTRs [mSMXL5 5′ UTR(1), mSMXL5 5′ UTR(2) or Scrambled SMXL5 5′ UTR(1-3)] were prepared using a QuickChange Site-Directed Mutagenesis Kit (Stratagene, USA). Subsequently, to prepare transgenic plant bodies, the full-length cDNA sequences of JUL1, JUL2 and JUL1 point mutations were cloned into a pCB302ES vector containing a 35S promoter and a HA epitope tag, and then the constructs were transformed into Agrobacterium tumefaciens GV3101, and Arabidopsis thaliana plants were transformed using a floral dipping method.

1-3. Transient Expression in Arabidopsis Protoplasts

Mesophyll protoplasts and plasmid DNA were prepared, and then 2×10⁴ protoplasts were transfected with 20 μg of the plasmid DNA and incubated at room temperature for 6 hours. In addition, for a reporter assay, 2×10⁴ protoplasts were transfected with 20 μg of total plasmid DNA consisting of different combinations of a reporter [SMXL5 5′ UTR-LUC, mSMXL5 5′ UTR(1)-LUC, mSMXL5 5′ UTR(2)-LUC, SMXL4 5′ UTR-LUC SMXL5 5′ UTR-GFP, mSMXL5 5′ UTR(1)-GFP, mSMXL5 5′ UTR(2)-GFP, or Scrambled SMXL5 5′ UTR-GFP(1-3)], an effector (JUL1-HA or JUL1^{R20/80/146A}-HA) and p35S-Rennila.

For RNA-immunoprecipitation, protoplasts transfected with 40 μg of plasmid DNA consisting of SMXL5 5′ UTR-GFP or mSMXL5 5′ UTR(1)-GFP were co-transfected into protoplasts with or without JUL1-HA. All assays were conducted at least three times, and similar results were obtained from all experiments.

1-4. Virus-Induced Gene Silencing (VIGS)

After pTRV2-derived vectors were transformed into A. tumefaciens strain GV2260, A. tumefaciens having a pTRV1 or pTRV2 construct was incubated overnight at 28° C., harvested, and then resuspended in 10 mM MgCl₂ and 10 mM MES. Virulence was induced by adding 200 μM acetosyringone, and incubating at room temperature for 2 to 4 hours. Subsequently, A. tumefaciens cells containing pTRV1 or pTRV2 were mixed in a 1:1 ratio, and infiltrated into the leaves of 3-week-old tobacco plants. For VIGS in tomatoes, A. tumefaciens strain GV3101 was transformed with a pTRV2-SlJUL construct, and the strain mixture containing pTRV1 and pTRV2 was then infiltrated into cotyledons of 2-week-old tomato plants. Approximately 3 to 4 weeks after infiltration, the plants were used for experiments.

1-5. Phylogenetic Analysis

To investigate homologs of JULs and SMXLs in Viridiplantae, 23 known taxa with related genome sequences in public databases (EnsemblPlants release 36, Phytozome ver.12, Solgenomics ver.3.1, Klebsormidium nitens v1.1) were analyzed. Protein sequences of two JUL proteins and 8 SMXL proteins, respectively, in Arabidopsis thaliana were used as query for tBLASTn search, and sequences of homologs in the top 10 hits of e-value≤10¹⁰ were subjected to a phylogenomic pipeline. Arabidopsis thaliana input sequences were obtained from TAIR. The top 10 tBLASTn hits (sorted by e-value) from each query were analyzed manually from each taxon to create homologous candidates. Sequence alignments of JULs and SMXLs were created using ClustalO and MAFFT, respectively, under default settings.

For more accurate sequence alignment, trimA1 was used for protein sequence alignment. Based on the JTT matrix-based model, maximum likelihood (ML) trees were constructed, branch supports were estimated using an ultrafast bootstrap approximation approach with 1,000 bootstrap replicates (−bb 1000) using IQ-TREE.

The best amino acid substitution model for each alignment was selected with the Nearest-Neighbor-Interchange ML heuristic method.

1-6. Identification of SMXL in S. moellendorffii

A specific genomic scaffold region (GL377574:2177376-2180294) was designated as a putative ORF of an AtSMXL5 homolog (SmSMXL). Based on the sequence, the SmSMXL ORF was cloned by PCR of total cDNA extracted from S. moellendorffii (purchased from http://www.xplant.co.kr, Seoul). CDS of SmSMXL was confirmed by sequencing with gene-specific primers. In addition, SNPs were corrected by aligning the sequence to the known genome of S. moellendorffii.

Afterward, for BLAST analysis, the revised CDS of SmSMXL was translated into a protein sequence. The location of SmSMXL is GL377569: 293451-296918 (EnsemblPlant).

1-7. Histological Analysis

For observation with optical microscopy, 6-week-old stem samples were fixed in 3% glutaraldehyde diluted in a 0.1 M sodium phosphate buffer (pH 7.2) for 3 hours, and then rinsed twice with a 0.1 M sodium phosphate buffer before being dehydrated through acetone. Subsequently, tissue sections were infiltrated and embedded in Spurr's resin (Electron Microscopy Sciences, USA) at 65° C. for 48 hours. Afterward, 2-μm-thick tissue sections were made using a RM2265 microtome (Leica, Germany), stained with 0.025% toluidine blue, and observed using an Axioplan 2 microscope (Carl Zeiss, Germany). For native staining, samples were directly sectioned with a razor blade, stained with 0.05% toluidine blue for 1 minute, rinsed with distilled water for 30 seconds, mounted with 50% glycerol, and observed using an Axioplan 2 microscope. For transmission electron microscopic observation, fixed tissues were subjected to OsO4 prior to dehydration.

1-8. Quantitative RT-PCR

Total RNA was isolated from inflorescent stems of 6-week-old plants using the TRIzol reagent (Invitrogen), and a reverse transcription polymerase chain reaction (RT-PCR) was then performed using 1 μg of the isolated RNA, oligo(dT) primers and ImProm-II reverse transcriptase (Promega). qRT-PCR was carried out using a SYBR Premix ExTaq system (Takara Bio, Japan) according to the instructions provided for LightCycler 2.0 (Roche Life Science, USA), and PCR primer sequences used in this experiment are listed in Table 2 below.

TABLE 2

| | | | SEQ |
|---|---|---|---|
| | | | ID |
| Gene | Direction | Sequence (5′→ 3′) | NO |
| SEOR1 | Forward | TCTCACGGCCTTGGTTCATC | 5 |
| (AT3G01680) | Reverse | ATCGACCCCAACCAAGTTCC | 6 |
| NEN4 | Forward | CGGTAGGATTTGGGCAGGTC | 7 |
| (AT4G39810) | Reverse | CCAAGACCAAAATAGGCAGCC | 8 |
| HB8 | Forward | GCTCGCTGGATCTCTCACTC | 9 |
| (AT4G32880) | Reverse | TCTTGAAGTGCCACCAACGT | 10 |
| TDR | Forward | TCTCGTCGGAAAACCTTGCA | 11 |
| (AT5G61480) | Reverse | ACCACCGTCGACTCTGTTTC | 12 |
| IRX3 | Forward | CAAAGGGTCCTAAACGTCCA | 13 |
| (AT5G17420) | Reverse | ATGGATGATTGCCCAAATGT | 14 |
| XCP1 | Forward | TGGAGAAAGAAAGGCGCTGT | 15 |
| (AT4G35350) | Reverse | ATGAGACCTCCATTGCAGCC | 16 |
| TUB4 | Forward | GCTAATCCTACCTTTGGTGATC | 17 |
| (AT5G44340) | Reverse | CAGCTTTCCACGGAACACAG | 18 |
| GFP | Forward | AGCAAGGGCGAGGAGCTG | 19 |
| | Reverse | GCACGCCGTAGGTGAAGG | 20 |
| YFP | Forward | GCAAGGGCGAGGAGCTG | 21 |
| | Reverse | GCACTGCAGGCCGTAGC | 22 |

1-9. Recombinant Protein Purification

For the purification of GST-fused recombinant proteins, GST-fused JUL1, JUL2, JUL1^{R20A}, JUL1^{R80A}, JUL1^{R146A}, JUL1^{R20/80A}, JUL1^{R80/146A}, JUL1^{R20/146A} or JUL1^{R20/80/146A} were expressed in Escherichia coli BL21, and the bacterial cells were incubated in a 200 mL Luria broth medium at 37° C. until an optical density at 600 nm reached 0.8, and then further incubated with 0.5 mM isopropyl β-d-1-thiogalactopyranoide (IPTG) for 3 hours. Afterward, the recombinant GST-fused proteins were purified according to the manufacturer's protocol (GE Healthcare, USA).

1-10. SELEX Analysis

For aptamer selection, a synthetic ssDNA library of a random sequence of 30 nucleotides, which is flanked by two primer regions (5′-GATAATACGACTCACTATAGGGTTACCTAGGTGTAGATGCT-(N) 30-AAGTGACGTCTGAACTGCTTCGAA-3; T7 promoter underlined; PMID: 25689224), was designed for in vitro transcription and amplification. All DNA oligos were synthesized by Cosmo Genetech (Korea). The ssDNA library was amplified using i-pfu, a forward primer (5′-GATAATACGACTCACTATAGGGTTACCTAGGTGTAGATGCT-3′) and a reverse primer (5′-TTCGAAGCAGTTCAGACGTCACTT-3′). The amplification cycle was optimized through gel analysis, and the amplified dsDNA library was purified using a PCR purification kit (Qiagen, Germany). Afterward, the dsDNA library was transcribed using an AmpliScribe™ T7 High Yield Transcription Kit (Epicenter, USA) at 37° C. for 2 hours, and then digested with DNase I. An RNA library was purified using phenol-chloroform-isoamyl alcohol extraction, followed by ethanol precipitation and measurement of a concentration using NanoDrop 2000 (Thermo Scientific, USA). Before the library was incubated with a target protein, RNA pools were refolded in a binding buffer (20 mM HEPES [pH 7.4], 150 mM KCl and 5 mM MgCl₂) by heating at 80° C. for 5 minutes and slowly cooling at room temperature. The target protein fused with GST was immobilized on Pierce Glutathione magnetic agarose beads (Thermo Scientific) in an equilibration buffer (125 mM Tris-HCl, 150 mM KCl, 1 mM DTT, 1 mM EDTA [pH 7.4]). After washing several times with a binding buffer, the RNA library was incubated with the target protein immobilized on the beads at room temperature for 1 hour, while being slowly stirred.

Meanwhile, to determine the binding proportion of the RNA library, the amount of unbound RNA was quantified using NanoDrop. The beads were washed twice with 100 μl of a buffer, and the bound RNA library was eluted in 8M urea by heating it at 95° C. for 10 minutes. The eluted RNA pool was recovered by ethanol precipitation, and then reverse-transcribed using a GoScript™ Reverse Transcription System (Promega). The produced oligo was used for the next round of SELEX, and each round was repeated using the same procedure described above. After the third round, the RNA pool was incubated with a target protein which was not immobilized on the magnetic agarose beads for negative selection. During the selection, several parameters were altered and stringently controlled: specifically, the ratio of RNA to the protein increased from 0.5 to 2.5; the incubation time decreased from 1 hour to 30 minutes; the washing volume increased from 100 μl to 200 μl, and the number of washes increased from 2 to 4. The separated SELEX was performed up to 15 rounds for a JUL1 protein, the selected RNA pool was amplified by PCR using the forward and reverse primers, and then cloned into a pENTR/TOPO vector. Escherichia coli TOP10 cells (TOPO-TA Cloning Kit, Thermo Fisher Scientific) were transformed with the vector. The clones containing ssDNA were purified using a Miniprep Kit (GeneAll, Korea) and sequenced (Cosmo Genetech). To analyze the structural similarity of the selected RNAs, their secondary structures were investigated using a QGRS program (http://bioinformatics.ramapo.edu/QGRS/index.php).

1-11. Electrophoretic Mobility Shift Assay (EMSA)

For RNA-EMSA of a PP library, single-stranded RNA PP was prepared by linearizing a pcDNA3.1 plasmid with ApaI and filling the 3′ overhang with a DNA polymerase I (Klenow) fragment (New England Biolabs, USA). Subsequently, transcription was performed using RiboMAX Large Scale RNA Production System-T7 (Promega) in the presence of [α-32P] UTP (10 mCi·mL⁻¹), and the RNA probes were gel-purified by denaturing them by gel electrophoresis using a 6% urea-TBE gel. For RNA-EMSA with the SMXL5 5′ UTR, ssRNA oligonucleotides of SMXL5 5′ UTR G-quadruplex-forming regions and SELEX probes were synthesized. The ssRNA probes were labeled with [γ-³²P] ATP (10 mCi·mL⁻¹) by being incubated with T4 polynucleotide kinase (New England Biolabs) at 37° C. for 30 minutes, and the unlabeled radionucleotides were removed using a Illustra MicroSpin G-25 column (Amersham, USA). A GST, GST-JUL1, GST-JUL2, GST-JUL1^{R20A}, GST-JUL1^{R80A}GST-JUL1^{R146A}, GST-JUL1^{R20/80A}, GST-JUL1^{R80/146A}, GST-JUL1^{R20/146A} or GST-JUL1^{R20/80/146A} protein was incubated in a binding buffer (10 mM Tris-HCl [pH 8.0], 2.5% glycerol, 0.5 mM DTT, 50 μg·mL⁻¹ BSA, 100 mM KCl, 250 μM EDTA and 1 μg heparin) having 20,000 cpm of ssRNA probes at room temperature for 20 minutes. Afterward, the reaction mixture was separated on a 6% polyacrylamide gel diluted with a 0.5×TBE buffer, and the gel was visualized on a Phosphor screen using a Typhoon FLA 9000 PhosphorImager (GE Healthcare).

1-12. Circular Dichroism Assay

All CD spectra were obtained from a J-815 Spectropolarimeter (Jasco, USA) at 25° C. using quartz cuvettes with a length of 2.0 mm. Each spectrum was recorded over a wavelength range of 220 nm to 320 nm at a scanning speed of 50 nm·min⁻¹, and the final spectrum was the average of five scans of the same sample. The synthesized RNA oligonucleotides (5 μM) were gradually cooled from 95° C. to room temperature in a binding buffer (10 mM Tris-HCl [pH 8.0] and 1 mM MgCl₂, in the presence or absence of 100 mM KCl), thereby forming a G-quadruplex, and allowed to reach equilibrium prior to CD measurement. The renatured SMXL5 5′ UTR containing 100 mM KCl was combined with a 2.5 μM JUL1 protein, and incubated at a constant temperature for 30 minutes before CD measurement to observe the G-quadruplex structure of the RNA in the SMXL5:JUL1 complex. In addition, to eliminate the effect of proteins and buffers, the spectra containing the JUL1 protein were calibrated using 2.5 μM JUL1 in buffer. In addition, to prove that JUL1 has no significant influence on the spectrum of the RNA G-quadruplex, the spectra of 2.5 μM JUL1 alone were recorded.

1-13. RNA Immunoprecipitation

To verify the interaction between the SMXL5 5′ UTR and JUL1, SMXL5 5′ UTR-GFP was co-transfected into protoplasts with or without JUL1-HA. Subsequently, an RNA-protein complex in a protoplast lysate was extracted using an IP buffer (100 mM KCl, 2.5 mM MgCl₂, 10 mM Tris-HCl [pH 8.0], 10% glycerol, 0.5% NP-40, 1 mM DTT, 100 U·mL⁻¹ RNasin RNase inhibitor (Promega), 25 mM MG132) and a protease inhibitor cocktail for plant cell extraction [Sigma-Aldrich, USA]). After insoluble debris was removed by centrifugation at 4° C. and 13,000 g for 10 minutes, 300 μl of a cell extract was incubated with 1 μg of HA-antibody (Roche) for 2 hours on ice with occasional gentle mixing, and a 30 μl aliquot of the cell extract was stored at −80° C. During the experiment, protein G agarose magnetic beads (Bio-Rad Laboratories, USA) were washed three times with a buffer (100 mM KCl, 2.5 mM MgCl₂, 10 mM Tris-HCl [pH 8.0], 10% glycerol, 0.5% NP-40 and 100 U·mL⁻¹ RNasin RNase inhibitor), and then the anti-HA-labeled extract was incubated with 10 μl protein G agarose magnetic beads for 2 hours at 4° C. with continuous stirring. Subsequently, the beads were washed 8 times with 1 mL of a buffer. After elution with the TRIzol reagent, the immunoprecipitated RNA and protein were analyzed by qRT-PCR, and detected using a HRP-conjugated high-affinity anti-HA antibody.

1-14. Surface Interaction Assay

For visualization of the interaction between the RNA G-quadruplex and JUL1, 20 μl of 5 μM RNA probes were heated for 5 minutes at 95° C. in a structure buffer (10 mM Tris-HCl [pH 8.0], 100 mM KCl, 100 mM NaCl or 100 mM LiCl, and 1 mM MgCl₂), and then gradually cooled at 25° C. for 1 to 2 hours. Subsequently, glutathione-sepharose 4B beads (GE Healthcare) were washed twice with a structure buffer, and incubated with GST-JUL1 and GST-JUL1^{R20/80/146A} (1.0-2.5 μM) at 4° C. for 1 hour. Afterward, the protein-bead complex was washed twice with a structure buffer and its volume was increased to 20 μl. The cooled and structured RNA probes were added to protein-bead complexes and incubated for 10 minutes with occasional mixing, ThT or NMM was added to the RNA probe-protein-bead complexes to a final concentration of 4 μM. To visualize the G-quadruplex structure bound to the bead surface, the fluorescence of ThT and NMM on the bead surface was observed using a CFP filter (460-500 nm) and an RFP filter (630-690 nm), respectively.

1-15. Transient Expression in HEK293T Cells

HEK293T cells were cultured in a DMEM medium (Welgene, Korea) including 10% fetal bovine serum (FBS) under conditions of 37° C. and 5% CO₂, and the cells were separated using trypsin during subculturing. Plasmid transfection was conducted using Metafectene Pro (Biontex Laboratories, Germany) according to the manufacturer's instructions. More specifically, the cells were co-transfected with a JUL1 expression plasmid (500 ng or 1000 ng of pCMV10-JUL1 or pCMV10-JUL1^{R20/80/146A} vector) and a GFP reporter (1 μg of pCMV10-SMXL5 5′ UTR-GFP or pCMV10-mSMXL5 5′ UTR-GFP). Forty-eight hours after transfection, the cells were harvested and subjected to western blot analysis.

1-16. Sucrose Density Gradient Analysis

Polysome profiles were obtained from Col-0, JUL1/2 RNAi seedlings and Arabidopsis thaliana protoplasts expressing SMXL5 5′ UTR-GFP in the presence or absence of JUL-HA. Briefly, the cells were treated with 100 μg·mL⁻¹ cycloheximide for 30 minutes and lysed in a polysome lysis buffer (200 mM Tris-HCl [pH 8.0], 50 mM KCl, 25 mM MgCl₂, 0.1 mM DTT, 0.5% NP-40 and 100 μg·mL⁻¹ cycloheximide). Subsequently, for each sample, 250 μl of a cell extract was dissolved on a 5 to 45% sucrose gradient in a polysome buffer (200 mM Tris-HC [pH 8.0], 50 mM KCl, 25 mM MgCl₂ and 0.1 mM DTT) by ultracentrifugation for 3 hours at 30,000 rpm with a SW41Ti rotor. A 0.25 mL fraction was obtained by a gradient density fractionator (Brandel, USA) using upward displacement with 60% (w/v) sucrose at a flow rate of 0.5 mL·min⁻¹. In addition, continuous monitoring was performed at an absorbance of 254 nm using an Econo UV monitor (Bio-Rad Laboratories). Total RNA of each fraction was isolated using the TRIzol reagent, subjected to qRT-PCR, and a GFP or TUB4 mRNA transcript level in each fraction was calibrated with a total input mRNA level of GFP or TUB4.

1-17. Measurement of Dissociation Constant Using Fluorescence Assay

The equilibrium dissociation constant was measured using a standard fluorescence assay. All RNA samples were renatured in binding buffer by heating at 95° C. for 5 minutes, and gradually cooled to room temperature for 1 hour prior to use. Fluorescein (FAM)-modified RNA samples were diluted to various concentrations from 0 to 500 nM using 100 μl of a binding buffer, and then mixed with JUL1-immobilized magnetic beads, followed by incubation in the dark for 1 hour at room temperature with gentle shaking. After unbound RNA was washed twice with a binding buffer, the RNA-JUL1 complex was eluted with 100 μl of a binding buffer supplemented with 500 mM imidazole. The fluorescence intensity of the eluate was measured with a 528/20 nm emission filter (with 485/20 nm fluorescence filter) using a Synergy™ HT multi-detection microplate reader (BioTek, USA). A Kd value was calculated by fitting to a kinetic model, which is the “exponential decay 1” model of OriginPro 9.0 software (OriginLab, USA).

1-18. Confocal Analysis

For the co-localization analysis of the MS2 protein, the MS2 hairpin-fused SMXL5 5′ UTR and the JUL1 protein, GFP and mRFP fluorescence were visualized under a confocal microscope (LSM510, Carl Zeiss). GFP was excited using a 488-nm argon laser line, and mRFP used a 543 nm-wavelength HeNe laser line. Emission wavelengths between 500 nm and 520 nm were recorded for GFP fluorescence, and mRFP fluorescence was recorded between 580 nm and 645 nm. For the co-localization of GFP and mRFP, a reconstructed Z-stack series were rendered as 3D interactive graphics. For the cytosolic accumulation of the JUL1 protein, the samples were treated with 0.01% NaN₃ for 10 minutes. For ThT fluorescence detection in protoplasts, the samples were treated with 10 μM ThT for 10 minutes before excitation with 405-nm argon laser, and an emission wavelength between 450 nm to 490 nm was collected. The imaging of YFP-expressing roots was obtained using 514-nm argon laser, and emission was detected in a wavelength range of 520 to 540 nm.

1-19. CFDA Treatment and Measurement

Cotyledons of 1.5 cm-long seedlings (selected from 4-day-old seedlings) were cut, and then directly treated with 5 μM CFDA-SE (Thermo Fisher). After CFDA treatment, the seedlings were placed on a 5% agarose gel to prevent drying, CFDA fluorescence in the root tip was observed under a fluorescence microscope (Axioplan2) equipped with a GFP filter at 0, 2 and 3 minutes after CFDA treatment, and fluorescence intensities were quantified by ImageJ.

1-20. Reverse Transcriptase Stalling Assay

An in vitro RT-stop assay was adopted with several modifications of a known method. RNA templates for the assay were prepared using pre-annealed DNA containing a minimal T7 promoter. After in vitro transcription using RiboMAX™ Large Scale RNA production system-T7 (Promega), products were purified by PCI/CI purification and subjected to gel-filtration using a G25 column (GE Healthcare). Subsequently, 150 to 200 μg of template RNA dissolved in nuclease-free water was added until 8 μl in the presence or absence of 1 μl of 10 μM pyridostatin (PDS) so that a final concentration of LiCl, NaCl or KCl became 10 mM, and 1 μl of a 5 μM radio-labeled primer was added. Afterward, the mixture was pre-incubated at 25° C. for 20 minutes, heated at 70° C. for 3 minutes, and then incubated at 4° C. for 10 minutes for primer annealing to a template.

An RT reaction was performed with a 150 mM of LiCl, NaCl or KC condition. The final mixture was incubated at 25° C. for 10 minutes, and subsequently at 50° C. for 20 minutes for reverse transcription. Subsequently, to stop the RT reaction, 1 μl of 1N NaOH and a 2× formamide denaturing buffer were added, incubated at 95° C. for 2 minutes, and cooled on ice. RT products were separated on a 8M UREA denaturing gel, fixed with 20% ethanol and 5% acetic acid, and visualized with a Phosphor screen using a Typhoon FLA 9000 PhosphorImager (GE Healthcare).

### Example 2. Identification of JULGI as Negative Regulator of Phloem Differentiation

2-1. Identification of JUL Gene

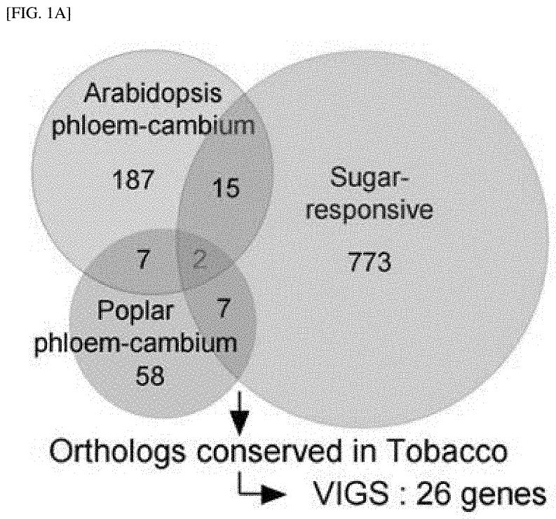

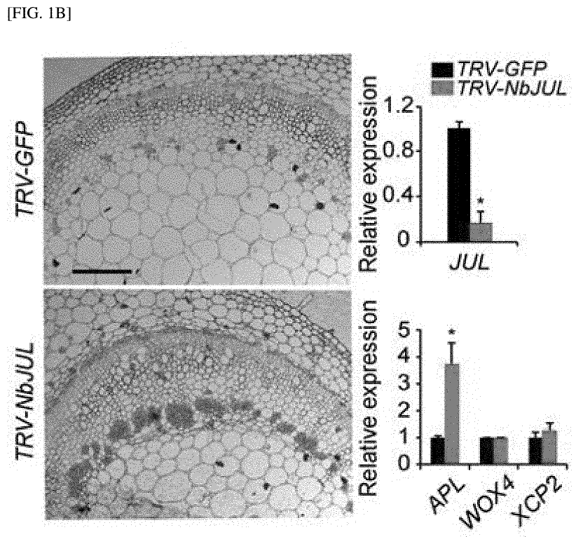

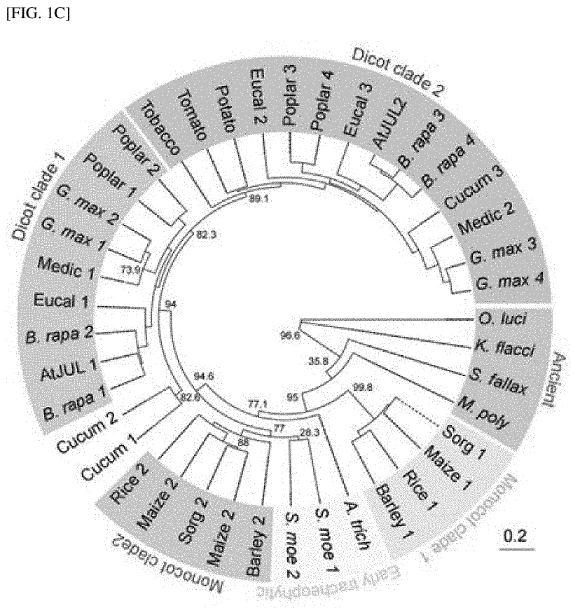

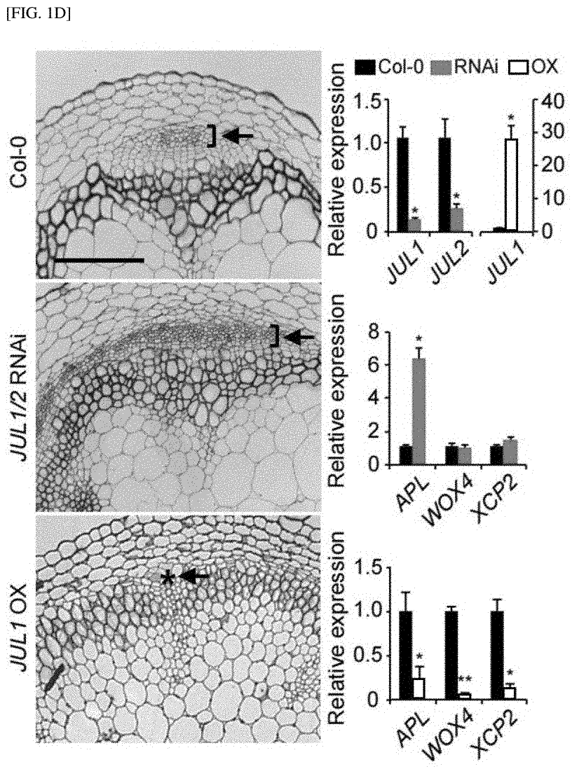

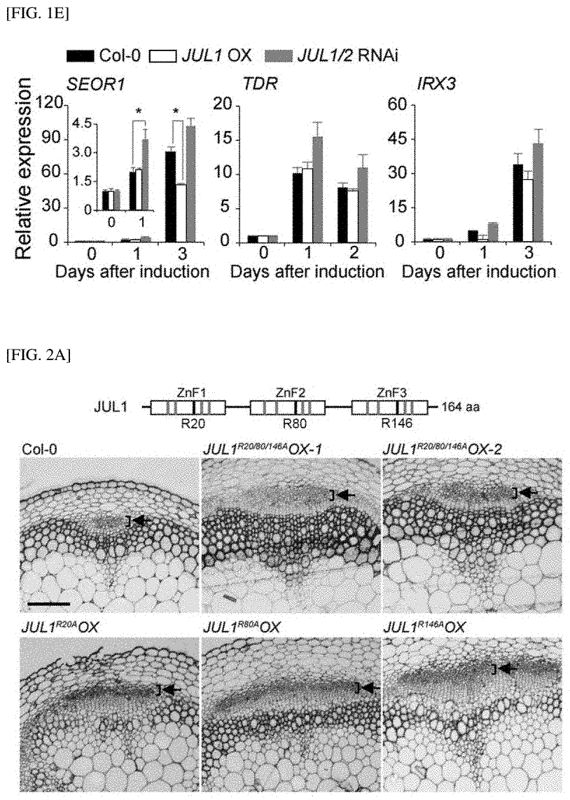

The inventors studied a fundamental mechanism of phloem formation in the evolution process of vascular plants, and here, hypothesized that carbon allocation throughout an entire plant body was optimized by regulating phloem differentiation by photosynthates, such as sugar. To verify the hypothesis, transcriptomes of the phloem/cambium region of a woody plant (Populus tremula), an herbaceous plant (Arabidopsis thaliana), and sucrose-regulated genes of Arabidopsis thaliana were comparatively analyzed. As a result, as shown in FIG. 1A, interestingly, it was confirmed that two sugar-regulated genes such as At3g15680 and At2g28790 are expressed even in the phloem/cambium of Arabidopsis thaliana and Populus tremula.

Next, the functionality of two candidates and 24 genes selected through computational and literature analysis as putative vascular regulators and controls were examined by virus-induced gene silencing (VIGS) in tobacco (Nicotiana benthamiana) according to the method described in Example 1-4. As a result, among the genes, the silencing (TRV-NbJUL) of a tobacco gene encoding a plant-specific protein having three RanBP2-type ZnF domains, such as Niben101Scf0620g02009 (At3g15680 ortholog), as shown in FIG. 1B, significantly increased the number of phloem cells and the expression of the phloem marker gene APL, and the expression of cambium (WOX4) or xylem (XYLEM CYSTEINE PEPTIDASE 2, XCP2) marker genes did not increase. The inventors named the novel regulator NbJULGI (NbJUL), which means “plant shoot” or “stream.”

2-2. Examination of Function of JUL Gene

Afterward, to investigate the role of JUL during the evolution of vascular plants, it was examined whether there are homologs of tobacco JUL in 23 species of various plants, including chlorophytes, charophytes, bryophytes, lycophytes, monocots and eudicots. As a result, as shown in FIG. 1C, it was confirmed that homologs are present in 15 vascular and 4 non-vascular plant species. In addition, phylogenetic analysis shows that homologs found in the 4-non-vascular plant species were branched from the outer group of vascular JUL homologs, which indicates that the non-vascular JUL homologs are ancient forms of vascular JUL proteins. Thus, the JUL proteins have diversified as vascular plants evolved, and more modern forms of the JUL proteins were present in both early vascular plants such as Selaginella and flowering plants, suggesting that JUL plays a critical role in the appearance of plant vasculature, particularly, the phloem. Indeed, silencing of JUL1 homologs in tomatoes (Solanum lycopersicum) and rice (Oryza sativa) increased the number of phloem cells, demonstrating an evolutionarily conserved role of the gene.

Further, to further elucidate the function of JUL in phloem formation, the effect of JUL1 (At3g15680) and JUL2 (At5g25490) after gene silencing in Arabidopsis thaliana was analyzed. As a result, it was confirmed that, when JUL1 or JUL2 expression is suppressed, there are slight variations in phloem cell population, and when the expression of JUL1 and JUL2 is completely suppressed (JUL1/2 RNAi), as shown in FIG. 1D, the degree of phloem differentiation and the expression of a phloem marker gene APL significantly increase, compared with the wild type (Col-0). In contrast, there was no change in expression of marker genes (WOX4 and XCP2) involved in xylem or vascular cambium development. In contrast, when JUL1-HA was overexpressed (JUL1 OX), severe abnormalities were generated in the phloem, xylem and cambium cell population in stems, and the expression of the APL, WOX4 and XCP2 genes also significantly decreased.

Based on the results, the inventors evaluated the role of JUL in formation of vascular tissue using a vascular cell induction culture system using Arabidopsis leaves (VISUAL) to quantify differentiation efficiency. As a result, as shown in FIG. 1E, it was confirmed that, when both JUL1 and JUL2 were silenced (JUL1/2 RNAi), on the first day of VISUAL induction, the expression of the phloem marker gene such as SIEVE ELEMENT OCCLUSION-RELATED 1 (SEOR1) was strongly induced, and there was no significant change in expression of cambium TDR (TDIF RECEPTOR) or xylem IRX3 (IRREGULAR XYLEM 3)-related genes. In addition, in contrast, it was confirmed that the overexpression of JUL1 (JUL1 OX) inhibited the induction of the expression of the phloem marker gene SEOR1 and the xylem marker gene IRX3 on day 3. Accordingly, from these results, it was seen that JUL serves as a negative regulator of evolutionarily conserved phloem differentiation in vascular plants.

### Example 3. Identification of G-Quadruplex Motif, as Target of JUL, in 5′ UTR of SMXL4/5

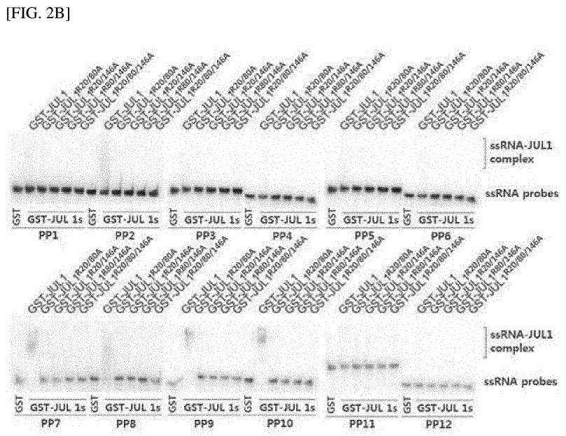

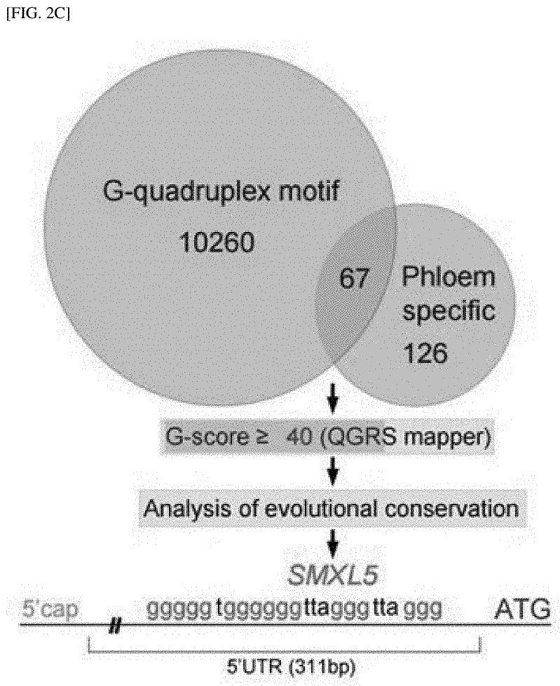

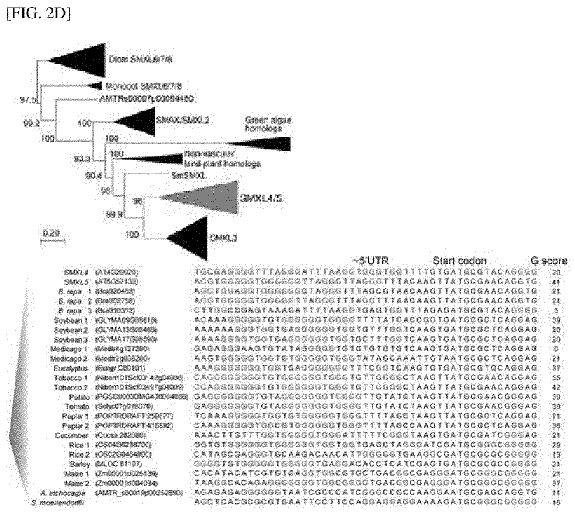

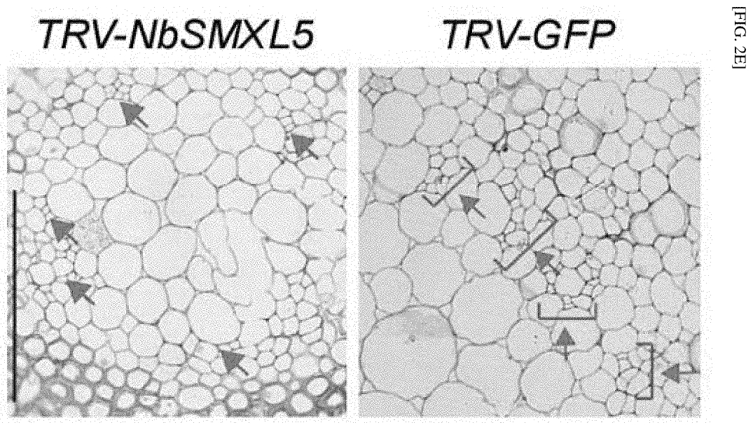

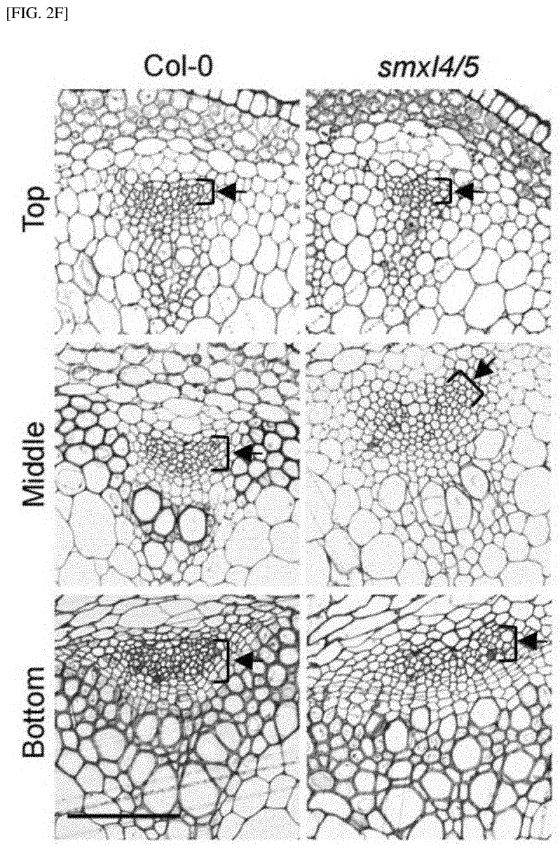

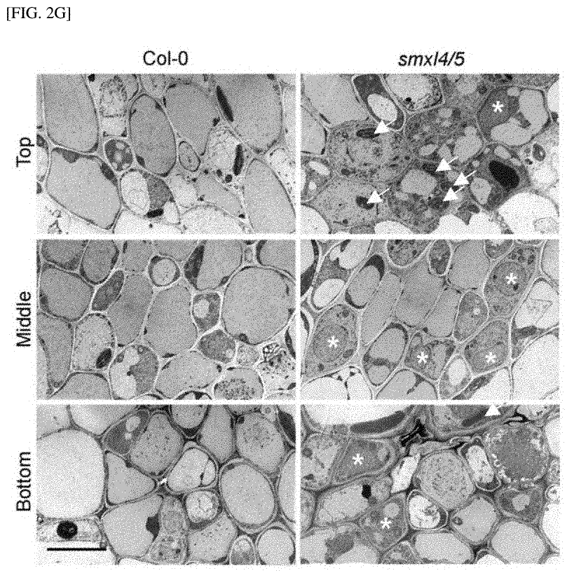

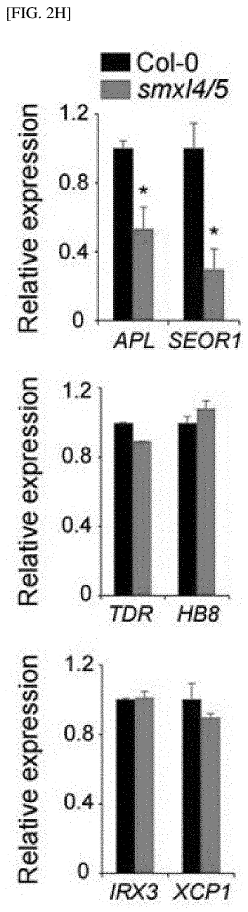

As shown in FIG. 2A, JUL1 and JUL2 have three RanBP2-type ZnF domains, each of which has a conserved arginine residue (R20, R80 and R146 in ZnF1, ZnF2 and ZnF3, respectively), which is required for RNA binding. Therefore, to examine whether ZnF domains of JUL1 are necessary for phloem differentiation, the inventors overexpressed four JUL1 mutants in which arginine was substituted with alanine, such as JUL1^{R20A}, JUL1^{R80A}, JUL1^{R146A}, and JUL1^{R20/80/146A}, and their effects were analyzed. As a result, as shown in FIG. 2A, similar to the results in the case of JUL1 and JUL2 silencing (JUL1/JUL2 RNAi), it was confirmed that all of the plant bodies expressing the mutants show significant increases in phloem cells, compared with the wild type (Col-0). These results indicate that ZnF domain mutants function as the main negative forms of JUL1 and by potentially interfering with the binding of proteins that interact with the wild-type JULs, it was demonstrated that the RNA binding activity of JUL1 is essential for its suppression of phloem development.

Afterward, whether JUL1 has a general RNA binding capability or sequence specificity was investigated using a random pentaprobe (PP) ssRNA library having penta-nucleotide diversity. To this end, glutathione S-transferase (GST)-fused JUL1 and its ZnF domain mutants were subjected to EMSA with an in vitro transcribed PP set according to the method described in Example 1-11. As a result, as shown in FIG. 2B, only a subset of PPs bound to JUL1, and JUL1 ZnF domain mutants (JUL1^{R20/80A}, JUL1^{R80/146A}, JUL1^{R20/146A} and JUL1^{R20/80/146A}) did not completely show PP mobility, suggesting that JUL1 can have sequence specificity for its target RNA.

Next, to identify the RNA target sequence recognized by JUL1, SELEX was performed with a random 30-nucleotide RNA library according to the method described in Example 1-10. As result of selectively concentrating and sequencing RNA probes bound to JUL1 up to 15 rounds, interestingly, it was found that JUL1-bound RNAs included guanine (G)-rich sequences, most of which formed G-quadruplexes (57 of 61 RNAs had a high G-score (>20)), and a secondary structure formed Hoogsteen-bonded G-quartet stacks. Further, to identify an in vivo mRNA target of JUL1, the inventors analyzed a set of 67 phloem-specific genes expected to have an RNA G-quadruplex-forming motif. As a result, as shown in FIG. 2C, through a computer scoring algorithm (G-score) based on the number of G-tetrads and the length of a loop connecting a G-tetrad, the 5′ UTR of SMXL5, which is known as a key positive regulator of phloem differentiation in Arabidopsis thaliana, was shown to have the highest possibility of G-quadruplex formation.

Based on this result, a phylogenetic analysis of the SMXL family showed that, as shown in FIG. 2D, SMXL3/4/5 are exclusively found in vascular plants and grouped separately from the other SMXL found in all plant species. In addition, the SMXL5 homolog of S. moellendorffii, which is an ancient vascular plant, is monophyletic, but grouped with SMXL3/4/5, implying that SmSMXL is an ancestor of SMXL3/4/5. Interestingly, a G-quadruplex-forming sequence was found in the 5′ UTR of 23 of 24 SMXL4/5 homologs and SmSMXL, whereas almost no G-quadruplex-forming motif was found in the 5′ UTR of other SMXLs in 23 sequenced plant species. Therefore, it was considered that the G-quadruplex-forming motif in the SMXL4/5 5′ UTR, which was exclusively conserved in vascular plants, probably serves as a specific substrate of JUL proteins for phloem development.

Here, the inventors analyzed phenotypes of smx14/5 function-lost mutants in tobacco and Arabidopsis thaliana. As a result, as shown in FIG. 2E, SMXL5 silencing in tobacco (TRV-NbSMXL5) resulted in a significant decrease in phloem differentiation. In addition, interestingly, compared with fully differentiated phloem of the wild-type vasculature, as shown in FIGS. 2F to 2H, it was confirmed that an smx14/5 Arabidopsis thaliana mutant had larger undifferentiated sieve elements still containing a nucleus and chloroplasts, and was significantly decreased in expression of phloem marker genes (APL and SEOR1). These results collectively demonstrate that SMXL4/5 5′ UTRs serve as a conserved target for phloem formation during the evolution of land plants.

### Example 4. Confirmation of G-Quadruplex Formation Through Direct Binding Between JUL and RNA G-Quadruplex Motif in SMXL5 5′UTR

4-1. EMSA

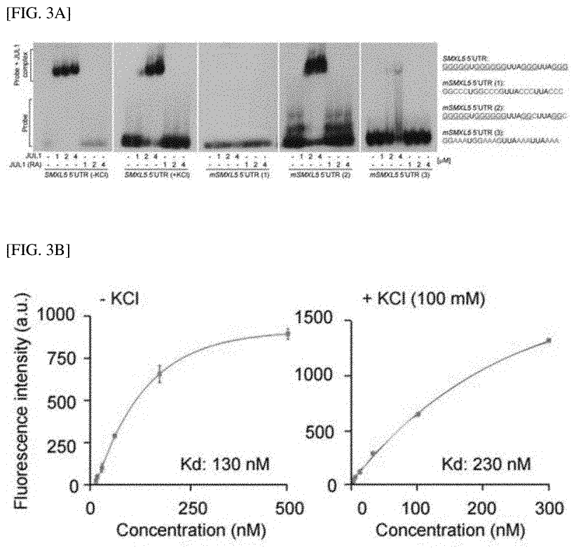

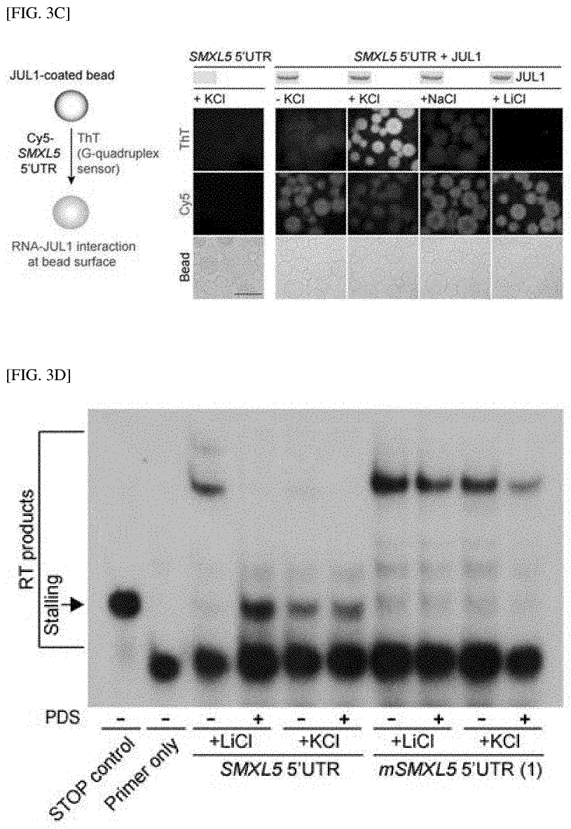

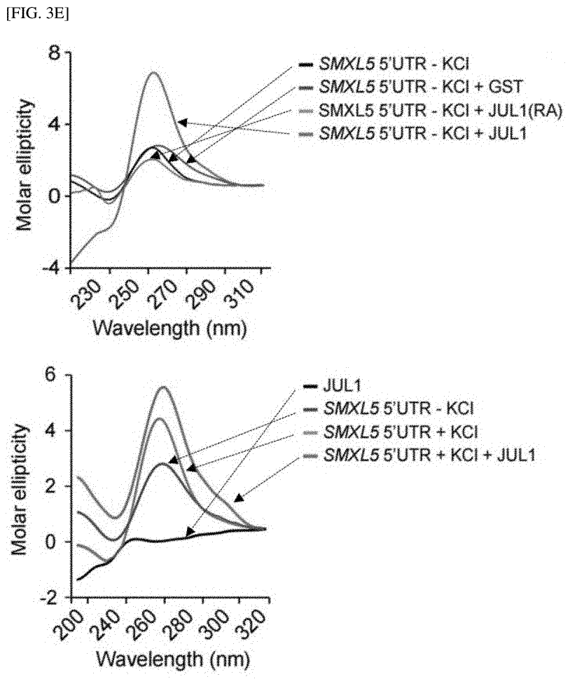

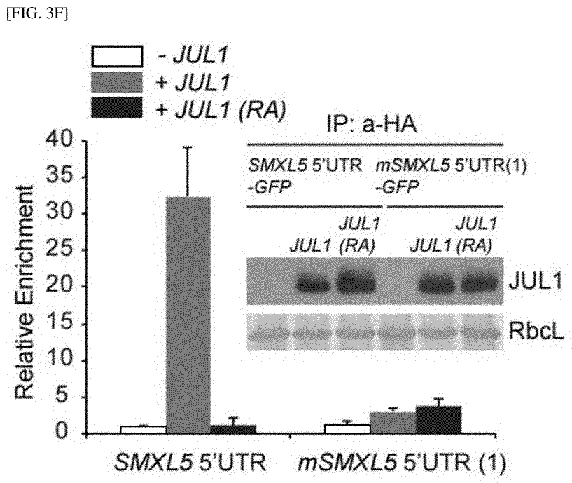

Based on the results described in Example 3, to examine whether JUL proteins directly bind to ssmRNA or the RNA G-quadruplex of SMXL5, EMSA was performed using GST-JUL1 and GST-JUL2 proteins in the presence or absence of potassium required for G-quadruplex formation. As result, as shown in FIG. 3A, it was confirmed that JUL1 delayed the mobility of the G-quadruplex-forming motif in the SMXL5 5′ UTR probe, which was used as a positive control. However, the mSMXL5 5′ UTR (1) and mSMXL5 5′ UTR (3), which had no G-quadruplex-forming motif, did not have the result described above. On the other hand, the mSMXL5 5′ UTR (2), which is a mutant producing two G-quartet layers, bound to JUL proteins less effectively than the SMXL5 5′ UTR. In addition, as a result of measuring the dissociation constant (Kd) of the binding between JUL1 and the SMXL5 5′ UTR, as shown in FIG. 3B, when there was no potassium, the Kd was estimated to be 130 nM, and when there was potassium, the Kd was estimated to be 230 nM, indicating that JUL1 preferentially binds to the primary SMXL5 5′ UTR sequence.

4-2. Surface Interaction Analysis

Next, the interaction between the SMXL5 5′ UTR G-quadruplex and JUL1 proteins was visualized using a surface binding system, which condenses RNA probes, on a protein-coated bead surface, with potassium strongly stabilizing a G-quadruplex, sodium weakly stabilizing a G-quadruplex, or lithium not stabilizing a G-quadruplex according to the method described in Example 1-14. To this end, as shown in FIG. 3C, a Cy5-labeled RNA probe, such as the SMXL5 5′ UTR, was treated with GST-JUL1-coated glutathione-sepharose beads, and the resulting G-quadruplex formed on the bead surface was simultaneously visualized using the Cy5 and G-quadruplex sensor, Thioflavin T (ThT).

As a result, as shown in FIG. 3C, the fluorescence signal emitted by ThT completely overlapped with the JUL1-coated bead surface in the presence of potassium (+KCl), but decreased in the presence of sodium (+NaCl) or lithium (+LiCl). Whereas, the Cy5 signal overlapping with the bead surface increased in the presence of sodium or lithium. These results suggest that direct binding of RanBP2-type ZnF of JUL1 to the G-quadruplex of the SMXL5 5′ UTR occurs. In addition, JUL1 bound to both the full-length of the SMXL5 5′ UTR and 5′ UTR fragments, which contain the G-quadruplex.

4-3. Reverse Transcriptase Stalling Analysis

In addition to the above results, to further confirm the formation of the RNA G-quadruplex, the structure of 68 bases of the SMXL5 5′ UTR having a U-rich region was investigated according to the method described in Example 1-20. As a result, as shown in FIG. 3D, in the presence of potassium (+KCl) or pyridostatin (PDS), which is a strong G-quadruplex-stabilizing ligand, reverse transcriptase stalling occurs in the SMXL5 5′ UTR, but did not occur in the mSMXL5 5′ UTR (1). Such stalling was observed only in the presence of potassium, not in the presence of lithium (+LiCl), and PDS strongly increased stalling in the presence of lithium. Such results suggest that the RNA G-quadruplex is formed in the 5′ UTR of SMXL5 rather than a G/U paired hairpin structure.

4-4. Measurement of Circular Dichroism (CD) Absorptivity

Next, the CD absorptivity of the G-quadruplex-forming motif in SMXL5 was measured according to the method described in Example 1-12. As a result, as shown in FIG. 3E, the molar ellipticity of the G-rich motif showed a negative peak at 240 nm and a positive peak at 264 nm, which is the signature spectrum of the RNA G-quadruplex. Interestingly, the incubation of JUL1 and the unstructured SMXL5 5′ UTR (SMXL5 5′ UTR+KCl+JUL1) increased the peak at 264 nm, suggesting that JUL1 induced folding of the G-quadruplex via direct binding to the SMXL5 5′ UTR. Moreover, the incubation of JUL1 with folded SMXL5 5′ UTR (SMXL5 5′ UTR+KCl+JUL1) also increased the peak at 264 nm, suggesting that the binding of JUL1 increases the formation of the folded SMXL5 5′ UTR. Therefore, these results showed that JUL1 primarily recognizes the single-stranded G-quadruplex-forming motif and potentially serves as an inducer of RNA G-quadruplex formation.

4-5. RNA Immunoprecipitation

Further, the inventors verified binding of JUL1 to the SMXL5 5′ UTR in vivo by performing RNA immunoprecipitation in protoplasts according to the method described in Example 1-13. As a result, as shown in FIG. 3F, it was confirmed that only the SMXL5 5′ UTR, not the mSMXL5 5′ UTR(1), was co-immunoprecipitated with haemagglutinin (HA)-tagged JUL1.

Collectively, these results suggest that JUL directly interacts with the 5′ UTR of SMXL5, and functions in G-quadruplex folding particularly in the 5′ UTR of SMXL5.

4-6. Analysis of Action of JUL on SMXL5

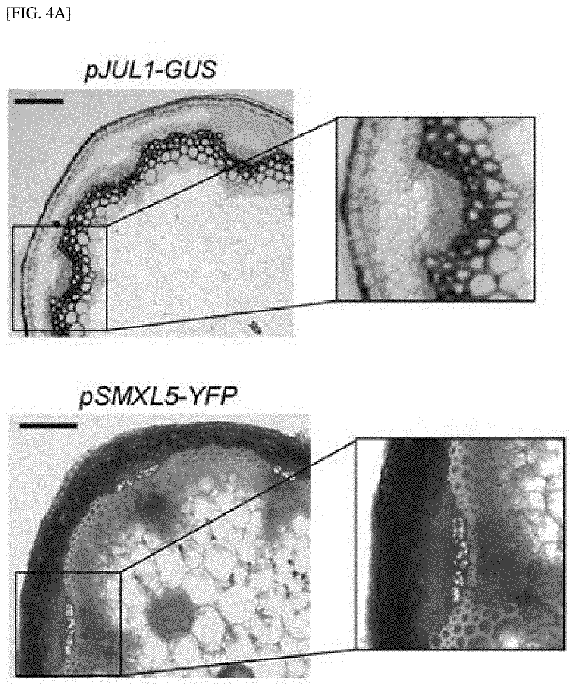

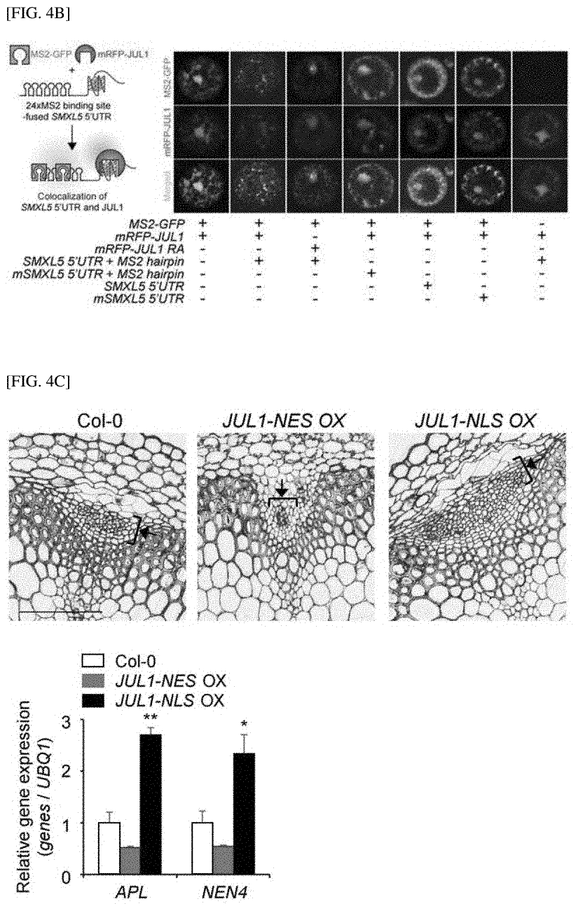

To investigate the JUL action on SMXL5, locations at which JUL and SMXL5 are expressed in the vascular tissue of the inflorescent stem were investigated using 0-glucuronidase (GUS) and yellow fluorescence protein (YFP) under the control of the JUL1 promoter and SMXL5 promoter, respectively. As a result, as shown in FIG. 4A, both JUL1(pJUL1-GUS) and SMXL5 (pSMXL-YFP) were specifically expressed in phloem and cambium regions of Arabidopsis thaliana stems, supporting the concept that JUL1 functions together with SMXL5 during phloem development. Interestingly, JUL1 was also expressed in pollen, vascular bundles of the roots, cotyledon and funicle.

Next, to examine whether JUL is bound with the G-quadruplex in the SMXL5 5′ UTR, cellular distribution of JUL1 and the SMXL5 5′ UTR was traced using a MS2 hairpin/MS2 coat protein-based RNA monitoring system in Arabidopsis thaliana protoplasts. Specifically, the SMXL5 5′ UTR conjugated to the 24×MS2 binding hairpin structure was directly visualized by GFP-tagged MS2 coat proteins. As a result, as shown in FIG. 4B, SMXL5 5′ UTR transcripts co-existed with JUL1 in Arabidopsis thaliana protoplasts, whereas the mSMXL5 5′ UTR and JUL1 RA did not co-exist with JUL1 and the SMXL5 5′ UTR, indicating that JUL1 is bound with the G-quadruplex motif of the SMXL5 5′ UTR in vivo.

In addition, to verify the function of JUL1 in phloem differentiation, the phenotypes of transgenic lines that overexpress JUL1 fused to a nuclear localization sequence (NLS) or a nuclear export sequence (NES) were observed. As a result, when JUL1-NES was overexpressed (JUL1-NES OX), the phenotype was similar to the overexpression phenotype of wild-type JUL1, and the expression of a phloem marker gene decreased, whereas when JUL1-NLS was overexpressed (JUL1-NLS OX), it was confirmed that phloem differentiation and the expression of corresponding marker genes (APL and NEN4) increased, similar to JUL1/2 RNAi and a JUL1 ZnF domain mutant in Examples 2-2 and 3.

### Example 5. Confirmation of Inhibition of SMXL5 Translation by JUL-Mediated Formation of G-Quadruplex

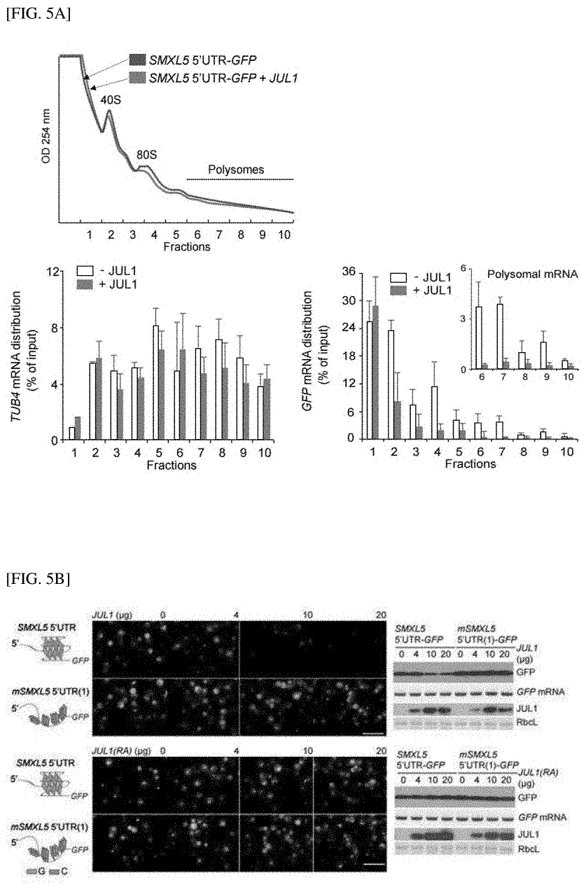

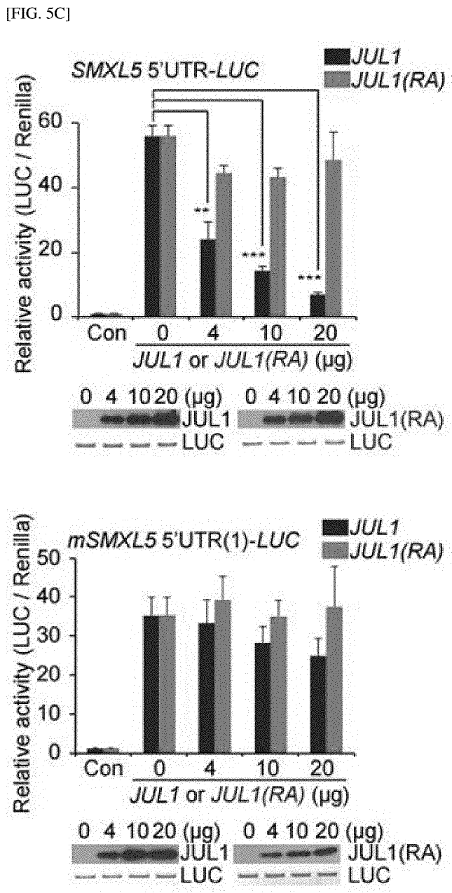

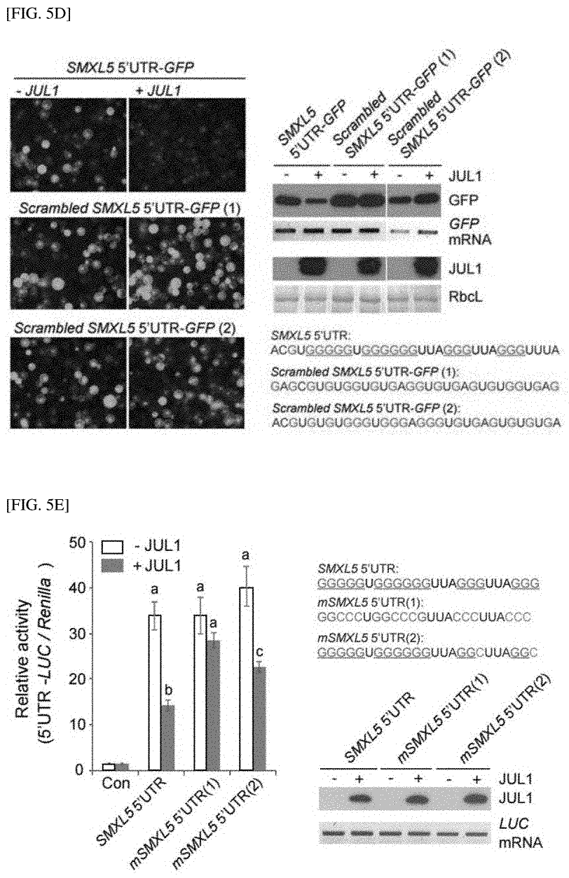

The 5′ UTR regulates translation in the cytosol, and the G-quadruplex typically imparts a translational inhibitory element onto the 5′ UTR. Therefore, the inventors hypothesized that cytosolic JUL1 regulates the expression of SMXL5, which is its specific target, through 5′ UTR G-quadruplex-mediated translational regulation. To elucidate the JUL1 action on SMXL5 mRNA, the ribosomal binding of SMXL5 in protoplasts was monitored. As a result, as shown in FIG. 5A, it was confirmed that, when JUL1 co-exists (SMXL5 5′ UTR-GFP+JUL1), the polysomal association of the SMXL5 5′ UTR-fused GFP mRNA dramatically decreased, whereas when TUB4 mRNA used as a control, the polysomal association did not decrease. In addition, in the case of the mSMXL5 5′ UTR (1) mutant, the inhibitory effect of JUL1 on the polysomal binding of GFP mRNA indicates that JUL1 suppresses the binding of SMXL5 transcripts to translationally activated ribosomes through 5′ UTR G-quadruplex recognition.

Next, to investigate whether JUL1 binding to the 5′ UTR G-quadruplex substantially affects the translation of SMXL5, GFP reporter analysis was performed under control of the SMXL5 5′ UTR or mutant 5′ UTRs [mSMXL5 5′ UTR (1)]. As a result, as shown in FIG. 5B, it was confirmed that the GFP signal and protein expression level under the control of the SMXL5 5′ UTR in the protoplasts were reduced by JUL1 in a dose-dependent manner, whereas the mSMXL5 5′ UTR (1) had no change in GFP signal and protein expression level, confirming no inhibitory effect of JUL1. In addition, in this case, it was confirmed that there is no change in expression of GFP mRNA regardless of the dose of JUL1. Consistent with these results, the reporter assay using SMXL4 or SMXL5 5′ UTR-fused luciferase (LUC) showed, as shown in FIG. 5C, a JUL1 concentration-dependent decrease in reporter activity, whereas translational suppression was not shown in the translation of the SMXL5 5′ UTR [mSMXL5 5′ UTR(1)] or JUL1 RA. In addition, as a result observed from SMXL5 5′ UTR mutants (Scrambled SMXL5 5′ UTR-GFP (1)/Scrambled SMXL5 5′ UTR-GFP (2)) that prevent G-quadruplex formation but have the same number of guanines, as shown in FIG. 5D, it was confirmed that, even in the presence of JUL1 (+JUL1), the expression of a GFP fluorescent protein was not reduced, confirming that JUL1-dependent translational suppression was not induced. In addition, in the presence of JUL1, the SMXL5′ UTR mutant with two layers of G-quartet (mSMXL5 5′ UTR (2)) had less of an inhibitory effect on the reporter activity than the SMXL5 5′ UTR, but its effect was higher than that of the single-stranded SMXL5 5′ UTR mutant (mSMXL5 5′ UTR(1)). These results suggest that direct recognition of the RNA G-quadruplex-forming motif and the stabilization of the G-quadruplex in the SMXL5 5′ UTR leads to the efficient JUL1-mediated suppression. Remarkably, as shown in FIG. 5E, the SMXL5 5′ UTR and its mutants did not show any difference in reporter activity in the absence of JUL1. These results indicate that JUL1 induces G-quadruplex formation in the SMXL5 5′ UTR, and stabilizes the G-quadruplex.

### Example 6. Confirmation of Control of Phloem Development by Negative Regulation of JUL-Mediated SMXL5

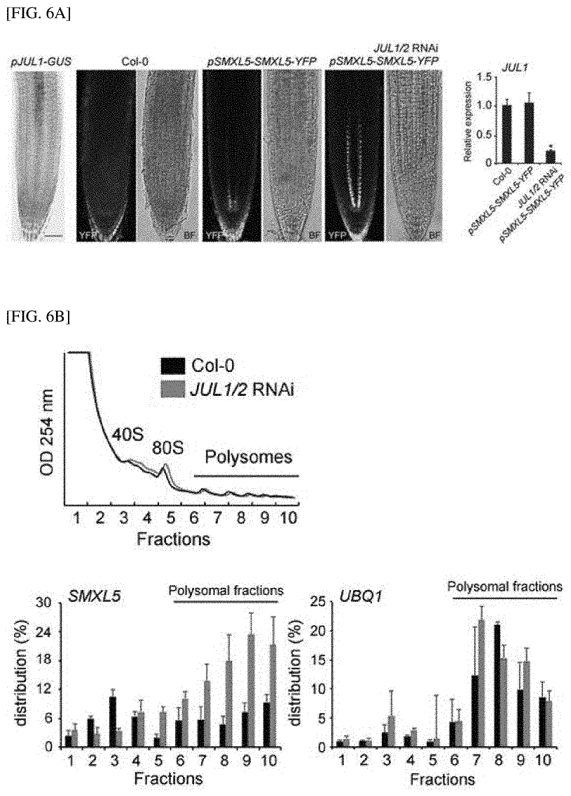

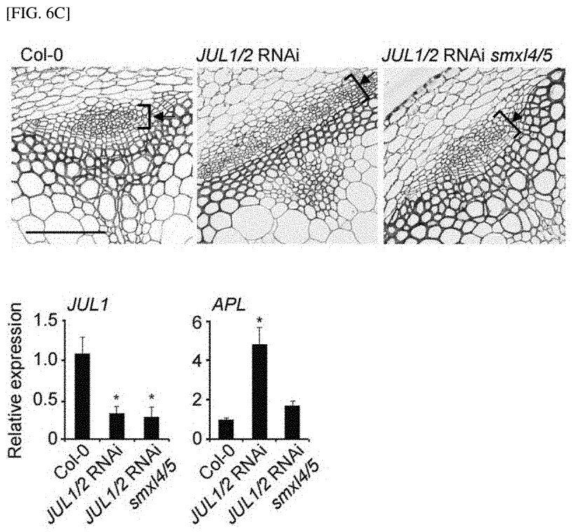

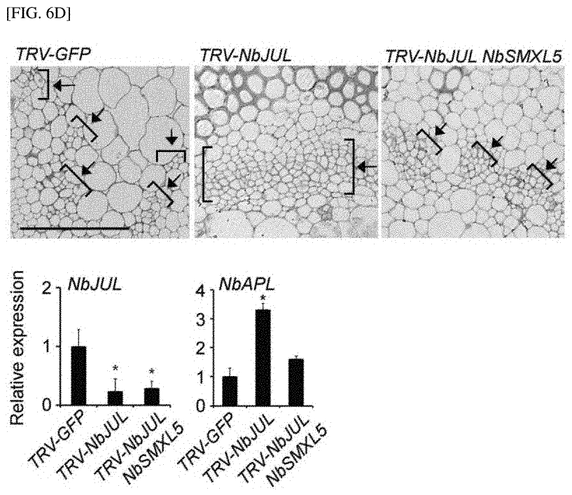

To investigate the effect of JUL-mediated SMXL5 5′ UTR G-quadruplex formation during phloem differentiation, the expression patterns of SMXL5 were observed under the control of the SMXL5 promoter in JUL1/2-silenced roots. As a result, as shown in FIG. 6A, the activity of the JUL1 promoter was limited to the elongation and maturation areas of roots, whereas SMXL5 was only detected in the early phloem cells of the basal meristem. However, when JUL1/2 silencing is induced (JUL1/2 RNAi), SMXL5 detection increased and expanded to the distal part of the basal meristem. In addition, as shown in FIG. 6B, when the JUL1/2 was silenced, the polysomal binding of SMXL5 to a plant body increased, indicating that the translation of SMXL5 increases by JUL1/2 silencing.

Further, to investigate the relationship between JUL and SMXL4/5 in phloem differentiation, the effect of defective SMXL4/5 was observed in JUL-silenced plants. As a result, as shown in FIG. 6C, the smx14/5 mutant reduced the expression of a phloem marker gene, APL, and partially restored the phenotype of the JUL1/2 RNAi line to wild-type. In addition, as shown in FIG. 6D, when NbSMXL5 was silenced by VIGS (TRV-NbJUL NbSMXL5), it was confirmed that the number of phloem cells in tobacco decreased, and the NbJUL-silenced phenotype was partially restored. These results supported that JUL functions upstream of SMXL4/5 in phloem development.

### Example 7. Confirmation of Control of Sink Strength of JUL-Mediated Vascular Plants

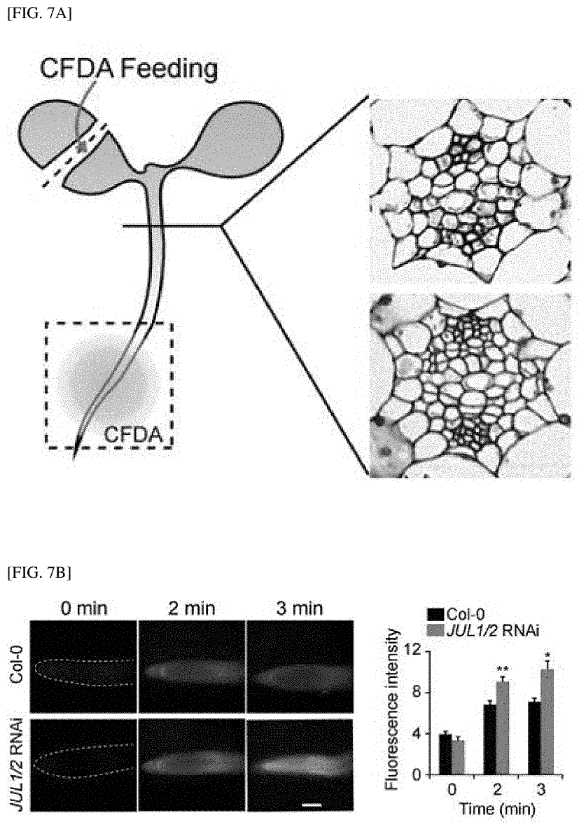

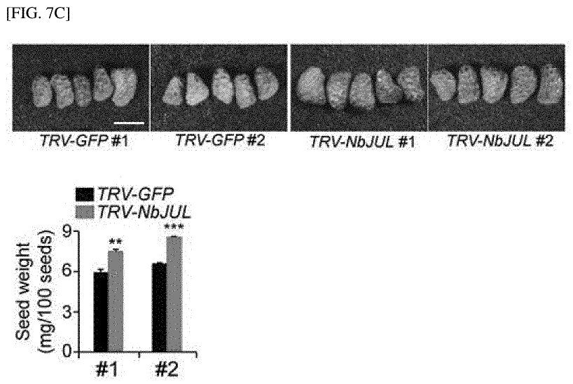

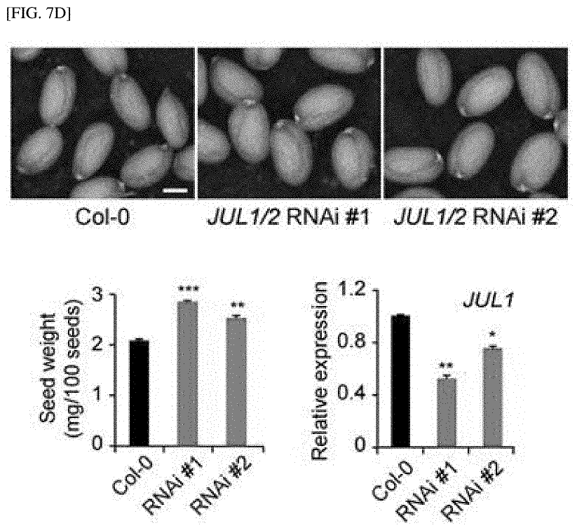

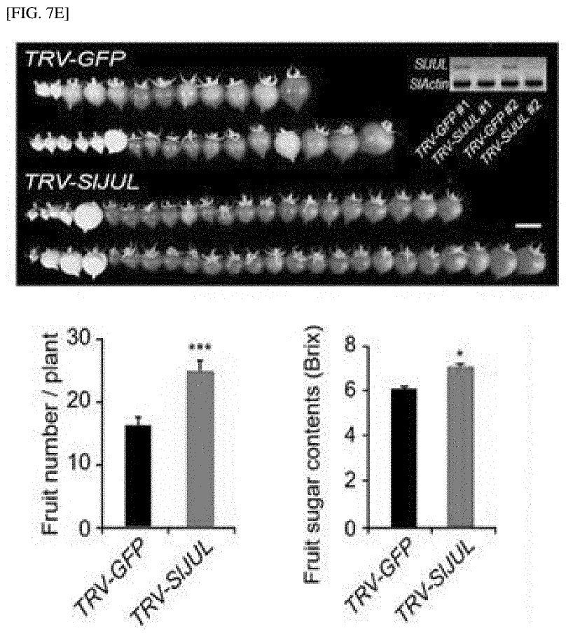

The inventors intended to discover whether the increased number of phloem cells relates to phloem transport capacity. To trace a phloem flow, as shown in FIG. 7A, the translocation of a phloem symplasmic tracer, such as 5, 6 carboxyfluorescein-diacetate (CFDA), from the cotyledon to the root tip was monitored. As the experimental result, as shown in FIG. 7B, when JUL1/2 was silenced (JUL1/2 RNAi), the number of phloem cells increased in the hypocotyl area, and CFDA accumulation in the root tip significantly increased, compared with a wild-type (Col-0) control, indicating that the phloem transport capacity is improved in JUL1/2 RNAi lines.

Next, JUL-deficient phenotypes of sink tissues such as seeds in tobacco and Arabidopsis thaliana were investigated. As a result, as shown in FIG. 7C, when NbJUL was silenced in tobacco (TRV-NbJUL #1, TRV-NbJUL #2), the seed size and weight increased approximately 26 to 29%, compared with controls (TRV-GFP #1, TRV-GFP #2). In addition, as shown in FIG. 7D, similar to JUL-deficient tobacco, in JUL1/2 RNAi Arabidopsis thaliana, compared with the wild-type (Col-0), it was confirmed that the total seed yield was not changed, but the seed size and weight increased by 37% and 22%, respectively. Moreover, the increase in seed size and weight is associated with the degree of JUL1 silencing in each RNAi line, indicating that there is a positive correlation between the increase in the number of phloem cells and the enhanced sink strength per seed. In addition, as a result of suppressing JULGI expression in tomatoes using VIGS, as shown in FIG. 7E, it was confirmed that the total yield of tomato flesh significantly increased, and a sugar content also significantly increased.

### Example 8. Confirmation of Control of Sink Strength of JUL-Mediated Vascular Plants Using Gene Editing

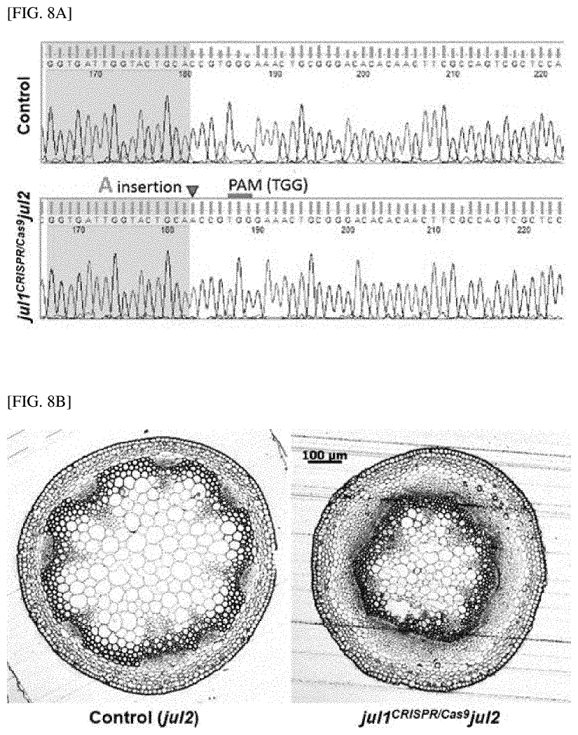

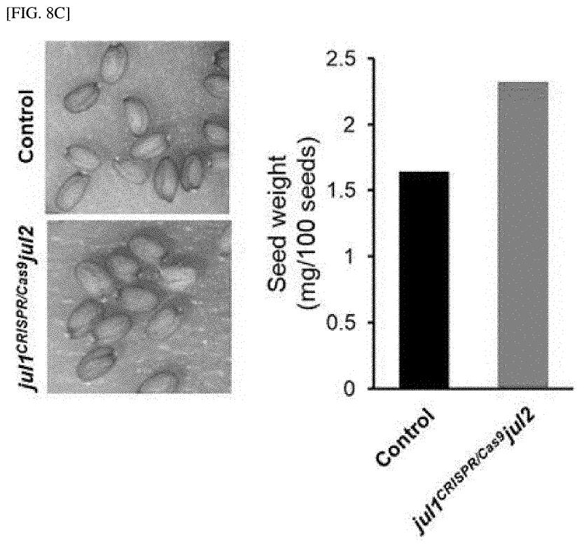

In addition to the result described in Example 7, using a type of gene editing, that is, a CRISPR/Cas9 system, as shown in FIG. 8A, a single nucleotide insertion was induced in a 5′ adjacent nucleic acid sequence of the JUL1 gene to knock-out the Arabidopsis thaliana JUL1 gene. As a result, as shown in FIG. 8B, compared with a control (Control(jul2)), it was confirmed that the phloem highly expands in a JUL1-knockout group (jul^{CRISPR/Cas9}jul2), and as shown in FIG. 8C, it was observed that the seed size and weight were also increased by approximately 40%. This means that the present invention can be effectively used with gene editing.

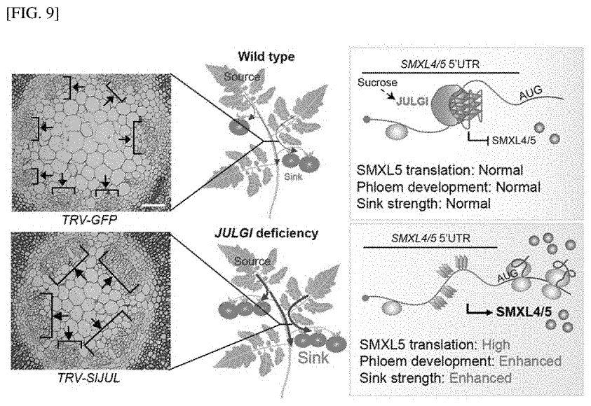

Collectively, as shown in FIG. 9, the correlation between the improvement in phloem development by JUL deficiency and the increase in sink strength of a sink organ supports the concept that phloem treatment capacity is directly associated with sink activity.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

In the present invention, when the expression or activity of a JULGI protein is suppressed, it has been confirmed that the phloem development in plant bodies is enhanced, and thus the sink strength of sink tissues such as seeds and fruits increases. Therefore, a composition including an inhibitor of the expression or activity of a JULGI protein according to the present invention may increase the sink strength of a plant sink tissue, thereby improving the productivity of various crops, and compared with the conventional methods requiring gene introduction, the present invention provides a method of producing higher value-added crops, which are free of GMO-related issues and thus can be effectively used in corresponding industries.

1. A method of increasing the sink strength of a sink tissue in a plant, comprising:
treating a plant body with an inhibitor of the expression or activity of a JULGI protein or a protein having 80% or more homology with the protein.

2. The method of claim 1, wherein the sink tissue is a seed, fruit, flower, root or tuber.

3. The method of claim 1, wherein the JULGI protein consists of an amino acid sequence represented by SEQ ID NO: 1 or 2.

4. The method of claim 1, wherein the JULGI protein binds to the 5′ untranslated region (UTR) of mRNA of SUPPRESSOR OF MAX2 1-LIKE4/5 (SMXL4/5) to suppress SMXL4/5 expression.

5. The method of claim 4, wherein the suppression of SMXL4/5 expression is accomplished by inducing RNA G-quadruplex formation by binding of the JULGI protein to the 5′ UTR of SMXL4/5 mRNA.

6. The method of claim 1, wherein the inhibitor is a virus-induced gene silencing (VIGS) vector including a gene encoding a JULGI protein or a protein having 80% or more homology with the JULGI protein.

7. The method of claim 1, wherein the inhibitor is an RNAi vector including a gene encoding a JULGI protein or a protein having 80% or more homology with the JULGI protein.

8. The method of claim 1, wherein the inhibitor is a CRISPR/Cas9 vector editing a gene encoding a JULGI protein or a protein having 80% or more homology with the JULGI protein.

9. The method of claim 6, wherein the gene encoding the JULGI protein consists of a nucleic acid sequence represented by SEQ ID NO: 3 or 4.

10. The method of claim 1, wherein the plant is selected from the group consisting of food crops including rice, wheat, barley, corn, peas, potatoes, red beans, oats, sorghum; vegetable crops including Arabidopsis, Chinese cabbage, radish, red pepper, strawberry, tomato, watermelon, cucumber, cabbage, melon, pumpkin, green onion, onion and carrot; special crops including Ginseng, tobacco, cotton, sesame, sugar cane, beet, Perilla, peanut and rapeseed; fruit trees including apple, pear, jujube, peach, kiwi, grape, tangerine, persimmon, plum, apricot and banana; flowers including roses, Gladiolus, Gerbera, carnations, Chrysanthemum, lilies and tulips; and forage and fodder crops including ryegrass, red clover, orchard grass, alfalfa, tall fescue and perennial ryegrass.

11. (canceled)

12. A plant body which is increased in sink strength of a sink tissue in a plant according to the method of claim 1.

13. The plant body of claim 12, wherein the plant is selected from the group consisting of food crops including rice, wheat, barley, corn, peas, potatoes, red beans, oats, sorghum; vegetable crops including Arabidopsis, Chinese cabbage, radish, red pepper, strawberry, tomato, watermelon, cucumber, cabbage, melon, pumpkin, green onion, onion and carrot; special crops including Ginseng, tobacco, cotton, sesame, sugar cane, beet, Perilla, peanut and rapeseed; fruit trees including apple, pear, jujube, peach, kiwi, grape, tangerine, persimmon, plum, apricot and banana; flowers including roses, Gladiolus, Gerbera, carnation, Chrysanthemum, lilies and tulips; and forage and fodder crops including ryegrass, red clover, orchard grass, alfalfa, tall fescue and perennial ryegrass.

14. The method of claim 7, wherein the gene encoding the JULGI protein consists of a nucleic acid sequence represented by SEQ ID NO: 3 or 4.

15. The method of claim 8, wherein the gene encoding the JULGI protein consists of a nucleic acid sequence represented by SEQ ID NO: 3 or 4.
